(19) United States
(12) Patent Application Publication
(10) Pub. No.: US 2025/0333471 A1
(43) Pub. Date: Oct. 30, 2025

(54) Chimeric Antigen Receptors Targeting HER2

(71) Applicant: City of Hope, Duarte, CA (US)

(72) Inventors: Saul J. Priceman, Valley Village, CA (US); Stephen J. Forman, Duarte, CA (US); Christine E. Brown, Altadena, CA (US)

(21) Appl. No.: 18/919,692

(22) Filed: Oct. 18, 2024

Related U.S. Application Data
(63) Continuation of application No. 17/543,148, filed on Dec. 6, 2021, now abandoned.
Application No. 17/543,148 is a continuation of application No. 15/773,754, filed on May 4, 2018, now Pat. No. 11,197,919.
(60) Provisional application No. 62/251,052, filed on Nov. 4, 2015.

(51) Int. Cl. C07K 14/705; A61K 39/00; A61K 40/11; A61K 40/31; A61K 40/42; A61K 40/50; A61P 35/00; C07K 14/71; C07K 14/725; C07K 16/28; C07K 16/32; C12N 5/0783; C12N 15/86

(52) U.S. Cl. CPC C07K 14/70521; A61K 40/11; A61K 40/31; A61K 40/4205; A61P 35/00; C07K 14/70503; C07K 14/7051; C07K 14/71; C07K 16/2863; C07K 16/32; C12N 5/0636; C12N 15/86; A61K 2039/505; A61K 40/50; C07K 2317/524; C07K 2317/622; C07K 2317/71; C07K 2319/03; C07K 2319/33; C12N 2740/16043; C12N 2830/15

(57) ABSTRACT

Chimeric transmembrane immunoreceptors (CAR) which include an extracellular domain targeted to HER2, a transmembrane region, a costimulatory domain and an intracellular signaling domain are described.

### CROSS-REFERENCE TO RELATED APPLICATIONS

This application is a continuation of U.S. application Ser. No. 17/543,148, filed on Dec. 6, 2021, which is a continuation of U.S. application Ser. No. 15/773,754, filed on May 4, 2018, now U.S. Pat. No. 11,197,919, which is the National Stage Application under 35 U.S.C. § 371 International Application No. PCT/US2016/060724, filed Nov. 4, 2016, which claims priority to and the benefit of U.S. Application No. 62/251,052, filed Nov. 4, 2015. The entire contents of the foregoing are incorporated herein by reference.

### SEQUENCE LISTING

This application contains a Sequence Listing that has been submitted electronically as an XML file named 40056-0021003.xml. The XML file, created on Oct. 17, 2024, is 49,000 bytes in size. The material in the XML file is hereby incorporated by reference in its entirety.

### BACKGROUND

Tumor-specific T cell based immunotherapies, including therapies employing engineered T cells, have been investigated for anti-tumor treatment. In some cases the T cells used in such therapies do not remain active in vivo for long enough periods. In some cases, the tumor-specificity of the T cells is relatively low, in part because of the heterogeneous nature of solid tumors and the potential for off-target effects on non-cancerous cells when targeting self antigens. Therefore, there is a need in the art for tumor-specific cancer therapies with improved anti-tumor specificity and function.

Chimeric antigen receptors (CARs) are composed of an extracellular tumor recognition/targeting domain, an extracellular linker/spacer, a transmembrane domain, and intracellular T cell-activating and co-stimulatory signaling domains. The design of the recognition/targeting domain is critical to avoiding deleterious off-target effects. The majority of CAR tumor targeting domains are single chain variable fragments (scFvs) derived from antibody sequences that exploit the specificity of antibody binding to particular antigens. There are also examples of CAR tumor targeting domains derived from normal receptor ligands, such as the IL-13 cytokine CAR that targets cells expressing the IL-13 receptor, IL13Rα2.

Adoptive T cell therapy (ACT) utilizing engineered T cells expressing a CAR has demonstrated robust and durable clinical efficacy in patients with CD 19+ B-cell malignancies (Priceman et al. 2015 Curr Opin Oncol; Maus et al. 2014 Blood 123:2625-2635. With early successes in hematologic diseases, broader application of this approach to solid tumors is now under intense investigation.

According to the National Cancer Institute Surveillance, Epidemiology, and End Results Program (SEER) data, there were an estimated 40,000 deaths from breast cancer in 2014, primarily from metastatic disease. Approximately 25-30% of breast cancer patients carry an amplification of the HER2 gene, which confers a particularly poor prognosis. Even with the advent of newer agents, including targeted therapies, there have been only modest improvements in overall mortality rates in Stage IV disease. For example, in a randomized trial with HER2-positive breast cancer patients, the most promising treatment combination of two HER2-targeted antibodies, trastuzumab and pertuzumab, plus docetaxel yields a median overall survival of 56.5 months and an extension of progression-free survival of only 6.3 months over trastuzumab and docetaxel alone.

### SUMMARY

Described herein are chimeric transmembrane immunoreceptors (chimeric antigen receptors or “CARs”) which comprise an extracellular domain, a transmembrane region and an intracellular signaling domain. The extracellular domain includes a scFv targeted to HER2 and, optionally, a spacer, comprising, for example, a portion of human Fe domain. The transmembrane portion includes, for example, a CD4 transmembrane domain, a CD8 transmembrane domain, a CD28 transmembrane domain, or a CD3 transmembrane domain. The intracellular signaling domain includes the signaling domain from the zeta chain of the human CD3 complex (CD3ξ) and one or more costimulatory domains, for example, a 4-IBB or a CD28 costimulatory domain. The extracellular domain enables the CAR, when expressed on the surface of a T cell, to direct T cell activity to those cells expressing HER2. Such cells include certain breast cancer cells and certain brain cancer cells. The inclusion of a costimulatory domain, such as the 4-IBB (CD137) costimulatory domain in series with CD3ξ in the intracellular region enables the T cell to receive co-stimulatory signals. T cells, for example, patient-specific, autologous T cells can be engineered to express the CARs described herein, and the engineered cells can be expanded and used in ACT. Various T cell subsets, including both alpha beta T cells and gamma delta T cells, can be used. In addition, the CAR can be expressed in other immune cells such as NK cells. Where a patient is treated with an immune cell expressing a CAR described herein the cell can be an autologous T cell or an allogenic T cell. In some cases the cells used are a cell population that includes both CD4+ and CD8+ central memory T cells (T_{CM}), which are CD62L+, CCR7+, CD45RO+, and CD45RA−, or the cells used are a cell population that includes CD4+ and CD8+ T_{CM} cells, stem central memory T cells and naive T cells (i.e., a population of T_{CM/SCM/N} cells). A population of T_{CM/SCM/N} cells are CD62L+, CCR7+ and include both CD45RA+ and CD45RO+ cells as well as both CD4+ cells and CD8+ cells. The use of such cells can improve long-term persistence of the cells after adoptive transfer compared to the use of other types of patient-specific T cells.

Described herein is a nucleic acid molecule encoding a CAR comprising: an scFv targeted to HER2 (e.g., DIQMTQSPSSLSASVGDRVTITCRASQDVNTAVAWYQQKPGKAPKLLIYSASFLY SGVPSRFSGSRSGTDFTLTISSLQPEDFATYYCQQHYTTPPTFGQGTKVEIKGSTSG GGSGGGSGGGGSSEVQLVESGGGL VQPGGSLRLSCAASGFNIKDTYIHWVRQAP GKGLEWVARIYPTNGYTRYADSVKGRFTISADTSKNTAYLQMNSLRAEDTAVY YCSRWGGDGFY AMDYWGQGTL VTVSS; SEQ ID NO: 1) or a variant thereof having 1-5 (e.g., 1 or 2) amino acid modifications (e.g., substitutions); a transmembrane domain selected from: a CD4 transmembrane domain or variant thereof having 1-5 (e.g., 1 or 2) amino acid modifications (e.g., substitutions), a CD8 transmembrane domain or variant thereof having 1-5 (e.g., 1 or 2) amino acid modifications (e.g., substitutions), a CD28 transmembrane domain or a variant thereof having 1-5 (e.g., 1 or 2) amino acid modifications (e.g., substitutions), and a CD3ξ transmembrane domain or a variant thereof having 1-5 (e.g., 1 or 2) amino acid modifications (e.g., substitutions); a costimulatory domain (e.g., a CD28 co-stimulatory domain or a variant thereof having 1-5 (e.g., 1 or 2) amino acid modifications (e.g., substitutions); or a 4-1BB co-stimulatory domain or a variant thereof having 1-5 (e.g., 1 or 2) amino acid modifications (e.g., substitutions); or both a CD28 co-stimulatory domain or a variant thereof having 1-5 (e.g., 1 or 2) amino acid modifications (e.g., substitutions) and a 4-1BB co-stimulatory domain or a variant thereof having 1-5 (e.g., 1 or 2) amino acid modifications (e.g., substitutions); and a CD3ξ signaling domain or a variant thereof having 1-5 (e.g., 1 or 2) amino acid modifications.

In various embodiments: the costimulatory domain is selected from the group consisting of: a CD28 costimulatory domain or a variant thereof having 1-5 (e.g., 1 or 2) amino acid modifications, a 4-1BB costimulatory domain or a variant thereof having 1-5 (e.g., 1 or 2) amino acid modifications and an OX40 costimulatory domain or a variant thereof having 1-5 (e.g., 1 or 2) amino acid modifications. In certain embodiments, a 4-1BB costimulatory domain or a variant thereof having 1-5 (e.g., 1 or 2) amino acid modifications in present. In some embodiments there are two costimulatory domains, for example a CD28 co-stimulatory domain or a variant thereof having 1-5 (e.g., 1 or 2) amino acid modifications (e.g., substitutions) and a 4-1 BB co-stimulatory domain or a variant thereof having 1-5 (e.g., 1 or 2) amino acid modifications (e.g., substitutions). In various embodiments the 1-5 (e.g., 1 or 2) amino acid modification are substitutions.

In some cases there is a short sequence of 1-6 amino acids (e.g. GGG) between the co-stimulatory domains and the CD3ξ signaling domain and/or between the two co-stimulatory domains.

Additional embodiment the CAR comprises: an scFv targeted to HER2; two different costimulatory domains selected from the group consisting of: a CD28 costimulatory domain or a variant thereof having 1-5 (e.g., 1 or 2) amino acid modifications, a 4-1 BB costimulatory domain or a variant thereof having 1-5 (e.g., 1 or 2) amino acid modifications and an OX40 costimulatory domain or a variant thereof having 1-5 (e.g., 1 or 2) amino acid modifications; two different costimulatory domains selected from the group consisting of: a CD28 costimulatory domain or a variant thereof having 1-2 amino acid modifications, a 4-1BB costimulatory domain or a variant thereof having 1-2 amino acid modifications and an OX40 costimulatory domain or a variant thereof having 1-2 amino acid modifications; a HER2 scFv or a variant thereof having 1-2 amino acid modifications; a transmembrane domain selected from: a CD4 transmembrane domain or variant thereof having 1-2 amino acid modifications, a CD8 transmembrane domain or variant thereof having 1-2 amino acid modifications, a CD28 transmembrane domain or a variant thereof having 1-2 amino acid modifications, and a CD3ξ transmembrane domain or a variant thereof having 1-2 amino acid modifications; a costimulatory domain (e.g., a CD28 co-stimulatory domain or a variant thereof having 1-5 (e.g., 1 or 2) amino acid modifications (e.g., substitutions); or a 4-1BB co-stimulatory domain or a variant thereof having 1-5 (e.g., 1 or 2) amino acid modifications (e.g., substitutions); or both a CD28 co-stimulatory domain or a variant thereof having 1-5 (e.g., 1 or 2) amino acid modifications (e.g., substitutions) and a 4-1BB co-stimulatory domain or a variant thereof having 1-5 (e.g., 1 or 2) amino acid modifications (e.g., substitutions); and CD3ξ signaling domain of a variant thereof having 1-2 amino acid modifications; a spacer region located between the HER2 scFv or variant thereof and the transmembrane domain (e.g., the spacer region comprises an amino acid sequence selected from the group consisting of SEQ ID NOs: 2-12 and 42 (Table 3) or a variant thereof having 1-5 (e.g., 1 or 2) amino acid modifications); the spacer comprises an IgG hinge region; the spacer region comprises 1-150 amino acids; there is no spacer; the 4-1 BB signaling domain comprises the amino acid sequence of SEQ ID N0:24 the CD3ξ signaling domain comprises the amino acid sequence of SEQ ID N0:21 and a linker of 3 to 15 amino acids that is located between the costimulatory domain and the CD3ξ signaling domain or variant thereof. In certain embodiments where there are two costimulatory domains, one is a 4-1BB costimulatory domain and the other a costimulatory domain selected from: CD28 and CD28gg. In various embodiments the 1-5 (e.g., 1 or 2) amino acid modification are substitutions, e.g., conservative substitutions.

In some embodiments: nucleic acid molecule expresses a polypeptide comprising an amino acid sequence selected from SEQ ID NOs: 26-41; the chimeric antigen receptor comprises an amino acid sequence selected from SEQ ID NOs: 26-41.

Also disclosed is a population of human T cells transduced by a vector comprising an expression cassette encoding a chimeric antigen receptor, wherein chimeric antigen receptor comprises: an scFv targeted to HER2; a transmembrane domain selected from: a CD4 transmembrane domain or variant thereof having 1-5 amino acid modifications (e.g., 1 or 2) amino acid modifications (e.g., substitutions), a CD8 transmembrane domain or variant thereof having 1-5 amino acid modifications (e.g., 1 or 2) amino acid modifications (e.g., substitutions), a CD28 transmembrane domain or a variant thereof having 1-5 amino acid modifications (e.g., 1 or 2) amino acid modifications (e.g., substitutions), and a CD3ξ transmembrane domain or a variant thereof having 1-5 amino acid modifications (e.g., 1 or 2) amino acid modifications (e.g., substitutions); a costimulatory domain (e.g., a CD28 co-stimulatory domain or a variant thereof having 1-5 (e.g., 1 or 2) amino acid modifications (e.g., substitutions); or a 4-1 BB co-stimulatory domain or a variant thereof having 1-5 (e.g., 1 or 2) amino acid modifications (e.g., substitutions); or both a CD28 co-stimulatory domain or a variant thereof having 1-5 (e.g., 1 or 2) amino acid modifications (e.g., substitutions) and a 4-1BB co-stimulatory domain or a variant thereof having 1-5 (e.g., 1 or 2) amino acid modifications (e.g., substitutions); and CD3ξ signaling domain of a variant thereof having 1-5 amino acid modifications (e.g., 1 or 2) amino acid modifications (e.g., substitutions). In various embodiments: the population of human T cells comprise a vector expressing a chimeric antigen receptor comprising an amino acid sequence selected from any of SEQ ID NOs: 26-41 or a variant thereof having 1-5 amino acid modifications (e.g., 1 or 2) amino acid modifications (e.g., substitutions); the population of human T cells comprises central memory T cells (T_{CM} cells) e.g., at least 20%, 30%, 40%, 50% 60%, 70%, 80% of the cells are T_{CM} cells, or the population of T cells comprises a combination of central memory T cells, naive T cells and stem central memory cells (T_{CM/SCM/N} cells) e.g., at least 20%, 30%, 40%, 50% 60%, 70%, 80% of the cells are T_{CM/SCM/N} cells. In either case, the population of T cells includes both CD4+ cells and CD8+ cells (e.g., at least 20% of the CD3+ T cells are CD4+ and at least 3% of the CD3+ T cells are CD8+ and at least 70, 80 or 90% are either CD4+ or CD8+; at least 15%, 20%, 25%, 30%, 35%, 40%, 50%, 60% of the cells CD3+ cells are CD4+ and at least 4%, 5%, 8%, 10%, 20 of the CD3+ cells are CD8+ cells).

Also described is a method of treating cancer in a patient comprising administering a population of autologous or allogeneic human T cells (e.g., autologous or allogenic T cells comprising central memory T cells (T_{CM} cells) or a combination of central memory T cells, naive T cells and stem central memory cells (i.e., the T cells are T_{CM/SCM/N} cells) at least 20%, 30%, 40%, 50% 60%, 70%, 80% of the cells are T_{CM/SCM/N} cells. In either case, the population of T cells includes both CD4+ cells and CD8+ cells (e.g., at least 20% of the CD3+ T cells are CD4+ and at least 3% of the CD3+ T cells are CD8+ and at least 70, 80 or 90% are either CD4+ or CD8+; at least 15%, 20%, 25%, 30%, 35%, 40%, 50%, 60% of the cells CD3+ cells are CD4+ and at least 4%, 5%, 8%, 10%, 20 of the CD3+ cells are CD8+ cells) transduced by a vector comprising an expression cassette encoding a chimeric antigen receptor, wherein chimeric antigen receptor comprises an amino acid sequence selected from SEQ ID NOs: 26-41 or a variant thereof having 1-5 (e.g., 1 or 2) amino acid modifications (e.g., substitutions). In various embodiments: the cancer is brain cancer, e.g., an HER2-expressing brain cancer that is a metastasis from breast cancer; and the transduced human T cells where prepared by a method comprising obtaining T cells from the patient, treating the T cells to isolate central memory T cells, and transducing at least a portion of the central memory cells to with a viral vector comprising an expression cassette encoding a chimeric antigen receptor, wherein chimeric antigen receptor comprises an amino acid sequence selected from SEQ ID NOs: 26 or 27 or a variant thereof having 1-5 (e.g., 1 or 2) amino acid modifications (e.g., substitutions). In some cases the CAR T cells are administered not directly the brain tumor, but are instead administered to the intraventricular space within the brain of the patient.

Also described is: a nucleic acid molecule encoding a polypeptide comprising an amino acid sequence that is at least 95% identical to an amino acid sequence selected from SEQ ID NOs 26-41; a nucleic acid molecule encoding an polypeptide comprising an amino acid sequence that is identical to an amino acid sequence selected from SEQ ID NOs: 26-4 1 except for the presence of no more than 5 amino acid substitutions, deletions or insertions; a nucleic acid molecule encoding an polypeptide comprising an amino acid sequence that is identical to an amino acid sequence selected from SEQ ID NOs: 26-41 except for the presence of no more than 5 amino acid substitutions; and a nucleic acid molecule encoding an polypeptide comprising an amino acid sequence that is identical to an amino acid sequence selected from SEQ ID NOs: 26-41 except for the presence of no more than 2 amino acid substitutions.

This disclosure also includes nucleic acid molecules that encode any of the CARs described herein (e.g., vectors that include a nucleic acid sequence encoding one of the CARs) and isolated T lymphocytes that express any of the CARs described herein.

The CAR described herein can include a spacer region located between the HER2 binding domain (e.g., a HER2 scFv) and the transmembrane domain. A variety of different spacers can be used. Some of them include at least portion of a human Fc region, for example a hinge portion of a human Fc region or a CH3 domain or variants thereof. Table 1 below provides various spacers that can be used in the CARs described herein.

TABLE 1
Examples of Spacers

| Name | Length | Sequence |
|---|---|---|
| a3 | 3 aa | AAA |
| linker | 10 aa | GGGSSGGGSG (SEQ ID NO: 2) |
| IgG4 hinge (S→P) | 12 aa | ESKYGPPCPPCP (SEQ ID NO: 3) |
| (S228P) | | |
| IgG4 hinge | 12 aa | ESKYGPPCPSCP (SEQ ID NO: 4) |
| IgG4 hinge | 22 aa | ESKYGPPCPPCPGGGSSGGGSG (SEQ |
| (S228P) + linker | | ID NO: 5) |
| CD28 hinge | 39 aa | IEVMYPPPYLDNEKSNGTIIHVKGKHL |
| | | CPSPLFPGPSKP (SEQ ID NO: 6) |
| CD8 hinge-48aa | 48 aa | AKPTTTPAPRPPTPAPTIASQPLSLRP |
| | | EACRPAAGGAVHTRGLDFACD (SEQ |
| | | ID NO: 7) |
| CD8 hinge-45aa | 45 aa | TTTPAPRPPTPAPTIASQPLSLRPEAC |
| | | RPAAGGAVHTRGLDFACD (SEQ ID |
| | | NO: 8 |
| IgG4(HL-CH3) | 129 aa | ESKYGPPCPPCPGGGSSGGGSGGQPRE |
| (includes | | PQVYTLPPSQEEMTKNQVSLTCLVKGF |
| S228P in hinge) | | YPSDIAVEWESNGQPENNYKTTPPVLD |
| | | SDGSFFLYSRLTVDKSRWQEGNVFSCS |
| | | VMHEALHNHYTQKSLSLSLGK SEQ |
| | | ID NO: 9) |
| IgG4 | 229 aa | ESKYGPPCPSCPAPEFEGGPSVFLFPP |
| (L235E, N297Q) | | KPKDTLMISRTPEVTCVVVDVSQEDPE |
| | | VQFNWYVDGVEVHQAKTKPREEQFSTY |
| | | RVVSVLTVLHQDWLNGKEYKCKVSNKG |
| | | LPSSIEKTISKAKGQPREPQVYTLPPS |
| | | QEEMTKNQVSLTCLVKGFYPSDIAVEW |
| | | ESNGQPENNYKTTPPVLDSDGSFFLYS |
| | | RLTVDKSRWQEGNVFSCSVMHEALHNH |
| | | YTQKSLSLSLGK (SEQ ID NO: |
| | | 10) |
| IgG4 | 229 aa | ESKYGPPCPPCPAPEFEGGPSVFLFPP |
| (S228P, L235E, | | KPKDTLMISRTPEVTCVVVDVSQEDPE |
| N297Q) | | VQFNWYVDGVEVHQAKTKPREEQFQST |
| | | YRVVSVLTVLHQDWLNGKEYKCKVSNK |
| | | GLPSSIEKTISKAKGQPREPQVYTLPP |
| | | SQEEMTKNQVSLTCLVKGFYPSDIAVE |
| | | WESNGQPENNYKTTPPVLDSDGSFFLY |
| | | SRLTVDKSRWQEGNVFSCSVMHEALHN |
| | | HYTQKSLSLSLGK (SEQ ID NO: |
| | | 11) |
| IgG4(CH3) | 107 aa | GQPREPQVYTLPPSQEEMTKNQVSLTC |
| | | LVKGFYPSDIAVEWESNGQPENNYKTT |
| | | PPVLDSDGSFFLYSRLTVDKSRWQEGN |
| | | VFSCSVMHEALHNHYTQKSLSLSLGK |
| | | (SEQ ID NO: 12) |
| HL | 22 aa | ESKYGPPCPPCPGGGSSGGGSG |
| | | (SEQ ID NO: 42) |

Some spacer regions include all or part of an immunoglobulin (e.g., IgG1, IgG2, IgG3, IgG4) hinge region, i.e., the sequence that falls between the CHI and CH2 domains of an immunoglobulin, e.g., an IgG4 Fe hinge or a CD8 hinge. Some spacer regions include an immunoglobulin CH3 domain or both a CH3 domain and a CH2 domain. The immunoglobulin derived sequences can include one or more amino acid modifications, for example, 1, 2, 3, 4 or 5 substitutions, e.g., substitutions that reduce off-target binding.

An “amino acid modification” refers to an amino acid substitution, insertion, and/or deletion in a protein or peptide sequence. An “amino acid substitution” or “substitution” refers to replacement of an amino acid at a particular position in a parent peptide or protein sequence with another amino acid. A substitution can be made to change an amino acid in the resulting protein in a non-conservative manner (i.e., by changing the codon from an amino acid belonging to a grouping of amino acids having a particular size or characteristic to an amino acid belonging to another grouping) or in a conservative manner (i.e., by changing the codon from an amino acid belonging to a grouping of amino acids having a particular size or characteristic to an amino acid belonging to the same grouping). Such a conservative change generally leads to less change in the structure and function of the resulting protein. The following are examples of various groupings of amino acids: 1) Amino acids with nonpolar R groups: Alanine, Valine, Leucine, Isoleucine, Proline, Phenylalanine, Tryptophan, Methionine; 2) Amino acids with uncharged polar R groups: Glycine, Serine, Threonine, Cysteine, Tyrosine, Asparagine, Glutamine; 3) Amino acids with charged polar R groups (negatively charged at pH 6.0): Aspartic acid, Glutamic acid; 4) Basic amino acids (positively charged at pH 6.0): Lysine, Arginine, Histidine (at pH 6.0). Another grouping may be those amino acids with phenyl groups: Phenylalanine, Tryptophan, and Tyrosine.

In certain embodiments, the spacer is derived from an IgG 1, IgG2, IgG3, or IgG4 that includes one or more amino acid residues substituted with an amino acid residue different from that present in an unmodified spacer. The one or more substituted amino acid residues are selected from, but not limited to one or more amino acid residues at positions 220, 226, 228, 229, 230, 233, 234, 235, 234, 237, 238, 239, 243, 247, 267, 268, 280, 290, 292, 297, 298, 299, 300, 305, 309, 218, 326, 330, 331, 332, 333, 334, 336, 339, or a combination thereof. In this numbering scheme, described in greater detail below, the first amino acid in the IgG4(L235E,N297Q) spacer in Table 1 is 219 and the first amino acid in the IgG4(HL-CH3) spacer in Table 1 is 219 as is the first amino acid in the IgG hinge sequence and the IgG4 hinge linker (HL) sequence in Table 1

In some embodiments, the modified spacer is derived from an IgG1, IgG2, IgG3, or IgG4 that includes, but is not limited to, one or more of the following amino acid residue substitutions: C220S, C226S, S228P, C229S, P230S, E233P, V234A, L234V, L234F, L234A, L235A, L235E, G236A, G237A, P238S, S239D, F243L, P247I, S267E, H268Q, S280H, K290S, K290E, K290N, R292P, N297A, N297Q, S298A, S298G, S298D, S298V, T299A, Y300L, V305I, V309L, E318A, K326A, K326W, K326E, L328F, A330L, A330S, A331S, P331S, I332E, E333A, E333S, E333S, K334A, A339D, A339Q, P396L, or a combination thereof.

In certain embodiments, the modified spacer is derived from IgG4 region that includes one or more amino acid residues substituted with an amino acid residue different from that present in an unmodified region. The one or more substituted amino acid residues are selected from, but not limited to, one or more amino acid residues at positions 220, 226, 228, 229, 230, 233, 234, 235, 234, 237, 238, 239, 243, 247, 267, 268, 280, 290, 292, 297, 298, 299, 300, 305, 309, 218, 326, 330, 331, 332, 333, 334, 336, 339, or a combination thereof.

In some embodiments, the modified spacer is derived from an IgG4 region that includes, but is not limited to, one or more of the following amino acid residue substitutions: 220S, 226S, 228P, 229S, 230S, 233P, 234A, 234V, 234F, 234A, 235A, 235E, 236A, 237A, 238S, 239D, 243L, 247I, 267E, 268Q, 280H, 290S, 290E, 290N, 292P, 297A, 297Q, 298A, 298G, 298D, 298V, 299A, 300L, 305I, 309L, 318A, 326A, 326W, 326E, 328F, 330L, 330S, 331S, 331S, 332E, 333A, 333S, 333S, 334A, 339D, 339Q, 396L, or a combination thereof, wherein the amino acid in the unmodified spacer is substituted with the above identified amino acids at the indicated position.

For amino acid positions in immunoglobulin discussed herein, numbering is according to the EU index or EU numbering scheme (Kabat et al. 1991 Sequences of Proteins of Immunological Interest, 5th Ed., United States Public Health Service, National Institutes of Health, Bethesda, hereby entirely incorporated by reference). The EU index or EU index as in Kabat or EU numbering scheme refers to the numbering of the EU antibody (Edelman et al. 1969 Proc Natl Acad Sci USA 63:78-85).

A variety of transmembrane domains can be used in the. Table 2 includes examples of suitable transmembrane domains. Where a spacer domain is present, the transmembrane domain is located carboxy terminal to the spacer domain.

TABLE 2
Examples of Transmembrane Domains

| Name | Accession | Length | Sequence |
|---|---|---|---|
| CD3z | J04132.1 | 21 aa | LCVLLDGILFIYGVILTAL |
| | | | FL (SEQ ID NO: 13) |
| CD28 | NM_006139 | 27 aa | FWVLVVVGGVLACYSLLVT |
| | | | VAFIIFWV (SEQ ID |
| | | | NO: 14) |
| CD28(M) | NM_006139 | 28 aa | MFWVLVVVGGVLACYSLLV |
| | | | TVAFIIFWV (SEQ ID |
| | | | NO: 15) |
| CD4 | M35160 | 22 aa | MALIVLGGVAGLLLFIGLG |
| | | | IFF (SEQ ID NO: 16) |
| CD8tm | NM_001768 | 21 aa | IYIWAPLAGTCGVLLLSLV |
| | | | IT (SEQ ID NO: 17) |
| CD8tm2 | NM_001768 | 23 aa | IYIWAPLAGTCGVLLLSLV |
| | | | ITLY (SEQ ID NO: |
| | | | 18) |
| CD8tm3 | NM_001768 | 24 aa | IYIWAPLAGTCGVLLLSLV |
| | | | ITLYC (SEQ ID NO: |
| | | | 19) |
| 41BB | NM_001561 | 27 aa | IISFFLALTSTALLFLLFF |
| | | | LTLRFSVV (SEQ ID |
| | | | NO: 20) |

Many of the CAR described herein include one or more (e.g., two) costimulatory domains. The costimulatory domain(s) are located between the transmembrane domain and the CD3ξ signaling domain. Table 3 includes examples of suitable costimulatory domains together with the sequence of the CD3ξ signaling domain.

TABLE 3
CD3 Domain and Examples of Costimulatory Domains

| Name | Accession | Length | Sequence |
|---|---|---|---|
| CD3ξ | 104132.1 | 113 aa | RVKFSRSADAPAYQQGQNQLYNEL |
| | | | NLGRREEYDVLDKRRGRDPEMGGK |
| | | | PRRKNPQEGLYNELQKDKMAEAYS |
| | | | EIGMKGERRRGKGEMGLYQGLSTA |
| | | | TKDTYDALHMQALPPR (SEQ ID |
| | | | NO: 21) |
| CD28 | NM 006139 | 42 aa | RSKRSRLLHSDYMNMTPRRPGPTR |
| | | | KHYQPYAPPRDFAAYRS (SEQ |
| | | | ID NO: 22) |
| CD28gg* | NM 006139 | 42 aa | RSKRSR@-QHSDYMNMTPRRPGPT |
| | | | RKHYQPYAPPRDFAAYRS (SEQ |
| | | | ID NO: 23) |
| 41BB | NM 001561 | 42 aa | KRGRKKLLYIFKQPFMRPVQTTQE |
| | | | EDGCSCRFPEEEEGGCEL (SEQ |
| | | | ID NO: 24) |
| OX40 | | 42 aa | ALYLLRRDQRLPPDAHKPPGGGSF |
| | | | RTPIQEEQADAHSTLAKI (SEQ |
| | | | ID NO: 25) |

### DESCRIPTION OF DRAWINGS

FIG. 1 depicts the amino acid sequence of Her2scFv-IgG4(S228P,L235E,N297Q)-CD28tm-CD28gg-Zeta-T2A-CD19t (SEQ ID NO: 26). The various domains are listed in order below the sequence and are indicated by alternating underlining and non-underlining. The mature CAR sequence (SEQ ID NO: 34) does not include the GMCSFRa signal peptide, the T2A skip sequence or truncated CD 19. Also called HER2(EQ)28ζ in FIGS. 3A-8E and throughout the specification.

FIG. 2 depicts the amino acid sequence of Her2scFv-IgG4(S228P,L235E,N297Q)-CD8tm-41BB-Zeta-T2A-CD 19t (SEQ ID NO:27). The various domains are listed in order below the sequence and are indicated by alternating underlining and non-underlining. The mature CAR sequence (SEQ ID NO: 35) does not include the GMCSFRa signal peptide, the T2A skip sequence or truncated CD 19. Also called HER2(EQ)BBζ in FIGS. 3A-8E and throughout the specification.

FIGS. 3A-D depict HER2-specific CAR constructs and CAR T cell expansion data.

FIGS. 4A-D depict in vitro characterization of HER2-CAR T cells against breast cancer cell lines.

FIGS. 5A-5F depict the result of studies on the in vitro tumor activity of HER2-CAR T cells.

FIGS. 6A-6I depict the result of studies on the in vivo anti-tumor efficacy of local intratumorally-delivered HER2-CAR T cells.

FIGS. 7A-7D depict the results of studies on local delivery of HER2-CAR T cells in human orthotopic BBM xenograft models.

FIGS. 8A-8E depict the results of studies on intraventrical delivery of HER2-CAR T cells.

FIG. 9 depicts the amino acid sequence of Her2scFv-CD8hinge-CD8tm-41BB-Zeta-T2A-CD19t (SEQ ID NO: 28). The various domains are listed in order below the sequence and are indicated by alternating underlining and non-underlining. The mature CAR sequence (SEQ ID NO: 36) does not include the GMCSFRa signal peptide, the T2A skip sequence or truncated CD 19.

FIG. 10 depicts the amino acid sequence of Her2scFv-IgG4hinge(S228P)-linker-CD8tm-41BB-Zeta-T2A-CD19t. (SEQ ID NO:29) The various domains are listed in order below the sequence and are indicated by alternating underlining and non-underlining. The mature CAR sequence (SEQ ID NO: 37) does not include the GMCSFRa signal peptide, the T2A skip sequence or truncated CD 19.

FIG. 11 depicts the amino acid sequence of Her2scFv-IgG4(S228P)-Linker-IgG4 CH3-CD8tm-41BB-Zeta-T2A-CD19t (SEQ ID NO: 30). The various domains are listed in order below the sequence and are indicated by alternating underlining and non-underlining. The mature CAR sequence (SEQ ID NO: 38) does not include the GMCSFRa signal peptide, the T2A skip sequence or truncated CD19.

FIG. 12 depicts the amino acid sequence of Her2scFv-IgG4(S228P)-Linker-IgG4 CH3-CD28tm-CD28gg-Zeta-T2A-CD19t (SEQ ID NO: 31). The various domains are listed in order below the sequence and are indicated by alternating underlining and non-underlining. The mature CAR sequence (SEQ ID NO: 39) does not include the GMCSFRa signal peptide, the T2A skip sequence or truncated CD19.

FIG. 13 depicts the amino acid sequence of Her2scFv-Linker-CD28tm-CD28gg-Zeta-T2A-CD19t (SEQ ID NO: 32). The various domains are listed in order below the sequence and are indicated by alternating underlining and non-underlining. The mature CAR sequence (SEQ ID NO: 40) does not include the GMCSFRa signal peptide, the T2A skip sequence or truncated CD19.

FIG. 14 depicts the amino acid sequence of Her2scFv-Linker-CD8tm-41BB-Zeta-T2A-CD19t. (SEQ ID NO: 33). The various domains are listed in order below the sequence and are indicated by alternating underlining and non-underlining. The mature CAR sequence (SEQ ID NO: 41) does not include the GMCSFRa signal peptide, the T2A skip sequence or truncated CD19.

FIGS. 15A-15C depict the results of studies characterizing certain additional CAR with various spacers. The IgG3(EQ) is in FIG. 2; DeltaCh2 is in FIG. 11; CD8h is in FIG. 9; HL is in FIG. 10; and L is in FIG. 14.

FIGS. 16A-16C show the results of studies examining CD107a and INF gamma produced when TCM expressing the various CAR are exposed to cells not expressing HER2 (MDA-MB-468), low HER2 (231BR), low HER2 (231BRHER2LO) or high HER2 (231BRHER2HI).

FIGS. 17A-17D show the results of studies examining PD-1 production and tumor cell killing ins various cell lines with the CAR of FIG. 2 (HER2(EQ)BBζ or FIG. 14 (HER2(L)BBζ).

FIGS. 18A-18B show the results of studies examining CD107a and INF gamma produced when TCM expressing the various CAR are exposed to cells not expressing HER2 (MDA-MB-468), low HER2 (231BR), low HER2 (231BRHER2LO) or high HER2 (231BRHER2HI).

### DETAILED DESCRIPTION

Described below is the structure, construction and characterization of various chimeric antigen receptors targeting HER2 and useful in treating HER2-expressing breast cancer as well as breast to brain metastasis. Importantly, the CAR described herein can be used in ACT to treat HER2 expressing tumors in the brain by intraventricular or intratumoral delivery.

A chimeric antigen (CAR) is a recombinant biomolecule that contains, at a minimum, an extracellular recognition domain, a transmembrane region, and an intracellular signaling domain. The term “antigen,” therefore, is not limited to molecules that bind antibodies, but to any molecule that can bind specifically to a target. For example, a CAR can include a ligand that specifically binds a cell surface receptor. The extracellular recognition domain (also referred to as the extracellular domain or simply by the recognition element which it contains) comprises a recognition element that specifically binds to a molecule present on the cell surface of a target cell. The transmembrane region anchors the CAR in the membrane. The intracellular signaling domain comprises the signaling domain from the zeta chain of the human CD3 complex and optionally comprises one or more costimulatory signaling domains. CARs can both to bind antigen and transduce T cell activation, independent of MHC restriction. Thus, CARs are “universal” immunoreceptors which can treat a population of patients with antigen-positive tumors irrespective of their HLA genotype. Adoptive immunotherapy using T lymphocytes that express a tumor-specific CAR can be a powerful therapeutic strategy for the treatment of cancer.

In some cases the CAR described herein can be produced using a vector in which the CAR open reading frame is followed by a T2A ribosome skip sequence and a truncated CD19 (CD19t), which lacks the cytoplasmic signaling tail (truncated at amino acid 323). In this arrangement, co-expression of CD19t provides an inert, non-immunogenic surface marker that allows for accurate measurement of gene modified cells, and enables positive selection of gene-modified cells, as well as efficient cell tracking and/or imaging of the therapeutic T cells in vivo following adoptive transfer. Co-expression of CD 19t provides a marker for immunological targeting of the transduced cells in vivo using clinically available antibodies and/or immunotoxin reagents to selectively delete the therapeutic cells, and thereby functioning as a suicide switch.

The CAR described herein can be produced by any means known in the art, though preferably it is produced using recombinant DNA techniques. Nucleic acids encoding the several regions of the chimeric receptor can be prepared and assembled into a complete coding sequence by standard techniques of molecular cloning known in the art (genomic library screening, PCR, primer-assisted ligation, site-directed mutagenesis, etc.) as is convenient. The resulting coding region is preferably inserted into an expression vector and used to transform a suitable expression host cell line, preferably a T lymphocyte cell line, and most preferably an autologous T lymphocyte cell line.

Various T cell subsets isolated from the patient, including unselected PBMC or enriched CD3 T cells or enriched CD3 or memory T cell subsets or T_{CM} or T_{CM/SCM/N} can be transduced with a vector for CAR expression. Central memory T cells are one useful T cell subset. Central memory T cell can be isolated from peripheral blood mononuclear cells (PBMC) by enriching for CD45RO+/CD62L+ cells, using, for example, the CliniMACS® device to immunomagnetically select cells expressing the desired receptors. The cells enriched for central memory T cells can be activated with anti-CD3/CD28, transduced with, for example, a SIN lentiviral vector that directs the expression of the CAR as well as a truncated human CD19 (CD19t), a non-immunogenic surface marker for both in vivo detection and potential ex vivo selection. The activated/genetically modified central memory T cells can be expanded in vitro with IL-2/IL-15 and then cryopreserved.

### Example 1: Structure of Two HER2-CAR

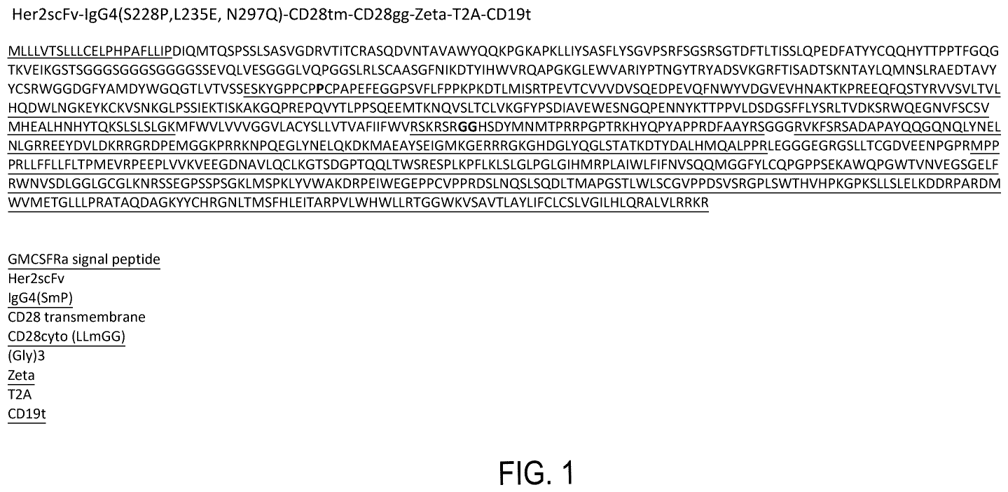

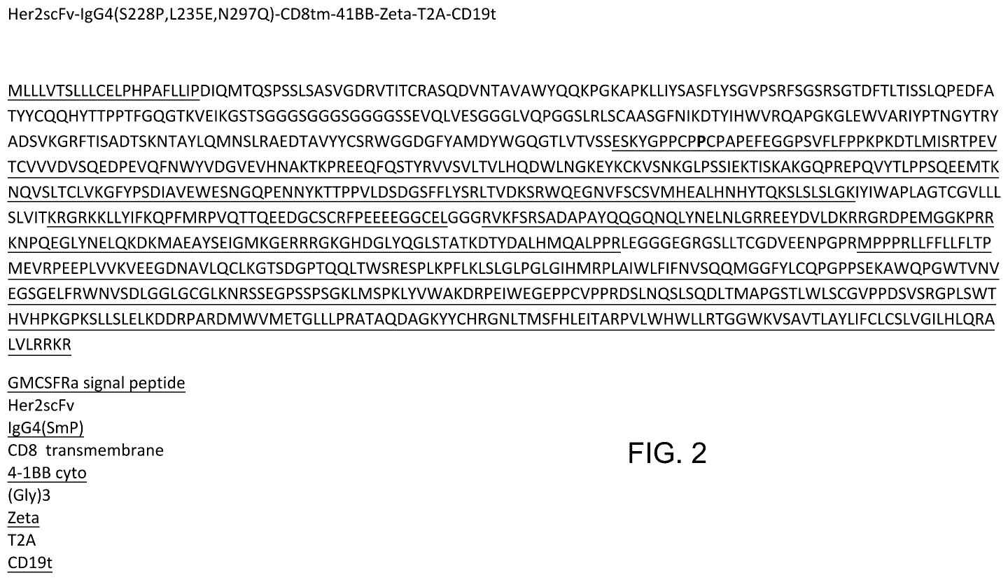

One CAR comprising a HER2 scFv described herein is referred to as Her2scFv-IgG4(L235E, N297Q)-CD28tm-CD28gg-Zeta-T2A-CD19t. This CAR includes a variety of important features including: a scFv targeted to HER2; an IgG4 Fe region that is mutated at two sites within the CH2 region (L235E; N297Q) in a manner that reduces binding by Fe receptors (FcRs); a CD28 transmembrane domain, a CD28 co-stimulatory domain, and CD3ξ activation domain. FIG. 1 presents the amino acid sequence of this CAR, including the sequence of the truncated CDI9 sequence used for monitoring CAR expression and the T2A ribosomal skip sequence that allows the CAR to be produced without fusion of the truncated CDI9 sequence. As shown in FIG. 2, the immature CAR includes: GMCSFR signal peptide, HER2 scFv, IgG4 that acts as a spacer, a CD8 transmembrane domain, a 4-IBB co-stimulatory domain that includes a LL to GG sequence alteration, a three Gly sequence, CD3 Zeta stimulatory domain. The transcript also encodes a T2A ribosomal sequence and a truncated CDI9 sequence that are not part of the CAR protein sequence. The mature CAR is identical to the immature CAR, but lacks the GMCSF signal peptide.

### Example 2: Construction and Structure of epHIV7 Used for Expression of HER2-Specific CAR T Cells

The epHIV7 vector is a vector that can used for expression of the HER2-specific CAR. Is was produced from pHIV7 vector. Importantly, this vector uses the human EFI promoter to drive expression of the CAR. Both the 5′ and 3′ sequences of the vector were derived from pv653RSN as previously derived from the HXBc2 provirus. The polypurine tract DNA flap sequences (cPPT) were derived from HIV-I strain pNL4-3 from the NIH AIDS Reagent Repository. The woodchuck post-transcriptional regulatory element (WPRE) sequence was previously described.

Construction of pHIV7 was carried out as follows. Briefly, pv653RSN, containing 653 bp from gag-pol plus 5′ and 3′ long-terminal repeats (LTRs) with an intervening SL3-neomycin phosphotransferase gene (Neo), was subcloned into pBluescript, as follows: In Step I, the sequences from 5′ LTR to rev-responsive element (RRE) made p5 ‘HIV-I 5I, and then the 5’ LTR was modified by removing sequences upstream of the TATA box, and ligated first to a CMV enhancer and then to the SV40 origin of replication (p5′HIV-2). In Step 2, after cloning the 3′ LTR into pBluescript to make p3′HIV-1, a 400-bp deletion in the 3′ LTR enhancer/promoter was made to remove cis-regulatory elements in HIV U3 and form p3′HIV-2. In Step 3, fragments isolated from the p5′HIV-3 and p3′HIV-2 were ligated to make pHIV-3. In Step 4, the p3′HIV-2 was further modified by removing extra upstream HIV sequences to generate p3′HIV-3 and a 600-bp BamHI-SalI fragment containing WPRE was added to p3′HIV-3 to make the p3′HIV-4. In Step 5, the pHIV-3 RRE was reduced in size by PCR and ligated to a 5′ fragment from pHIV-3 (not shown) and to the p3′HIV-4, to make pHIV-6. In Step 6, a 190-bp BglII-BamHI fragment containing the cPPT DNA flap sequence from HIV-I pNL4-3 (55) was amplified from pNL4-3 and placed between the RRE and the WPRE sequences in pHIV 6 to make pHIV-7. This parent plasmid pHIV7-GFP (GFP, green fluorescent protein) was used to package the parent vector using a four-plasmid system.

A packaging signal, psi (ψ), is required for efficient packaging of viral genome into the vector. The RRE and WPRE enhance the RNA transcript transport and expression of the transgene. The flap sequence, in combination with WPRE, has been demonstrated to enhance the transduction efficiency of lentiviral vector in mammalian cells.

The helper functions, required for production of the viral vector), are divided into three separate plasmids to reduce the probability of generation of replication competent lentivirus via recombination: 1) pCgp encodes the gag/pol protein required for viral vector assembly; 2) pCMV-Rev2 encodes the Rev protein, which acts on the RRE sequence to assist in the transportation of the viral genome for efficient packaging; and 3) pCMV-G encodes the glycoprotein of the vesiculo-stomatitis virus (VSV), which is required for infectivity of the viral vector.

There is minimal DNA sequence homology between the pHIV7 encoded vector genome and the helper plasmids. The regions of homology include a packaging signal region of approximately 600 nucleotides, located in the gag/pol sequence of the pCgp helper plasmid; a CMV promoter sequence in all three helper plasmids; and a RRE sequence in the helper plasmid pCgp. Itis highly improbable that replication competent recombinant virus could be generated due to the homology in these regions, as it would require multiple recombination events. Additionally, any resulting recombinants would be missing the functional LTR and tat sequences required for lentiviral replication.

The CMV promoter was replaced by the EFlα-HTLV promoter (EFIp), and the new plasmid was named epHIV7. The EFlp has 563 bp and was introduced into epHIV7 using NruI and NheI, after the CMV promoter was excised.

The lentiviral genome, excluding gag/pol and rev that are necessary for the pathogenicity of the wild-type virus and are required for productive infection of target cells, has been removed from this system. In addition, the vector construct does not contain an intact 3′LTR promoter, so the resulting expressed and reverse transcribed DNA proviral genome in targeted cells will have inactive LTRs. As a result of this design, no HIV-I derived sequences will be transcribed from the provirus and only the therapeutic sequences will be expressed from their respective promoters. The removal of the LTR promoter activity in the SIN vector is expected to significantly reduce the possibility of unintentional activation of host genes.

### Example 3: Production of Vectors for Transduction of Patient T Cells

Vectors for transduction of patient T cells can be prepared as follows. For each plasmid the plasmid expressing the CAR and, optionally, a marker such as truncated CD19; 2) pCgp; 3) pCMV-G; and 4) pCMV-Rev2), a seed bank is generated, which is used to inoculate the fermenter to produce sufficient quantities of plasmid DNA. The plasmid DNA is tested for identity, sterility and endotoxin prior to its use in producing lentiviral vector.

Briefly, cells are expanded from the 293T working cell (WCB), which has been tested to confirm sterility and the absence of viral contamination. A vial of 293T cells from the 293T WCB is thawed. Cells are grown and expanded until sufficient numbers of cells exists to plate an appropriate number of 10 layer cell factories (CFs) for vector production and cell train maintenance. A single train of cells can be used for production.

The lentiviral vector is produced in sub-batches of up to 10 CFs. Two sub-batches can be produced in the same week leading to the production of approximately 20 L of lentiviral supernatant/week. The material produced from all sub-batches is pooled during the downstream processing phase, in order to produce one lot of product. 293T cells are plated in CFs in 293T medium (DMEM with 10% FBS). Factories are placed in a 37° C. incubator and horizontally leveled in order to get an even distribution of the cells on all the layers of the CF. Two days later, cells are transfected with the four lentiviral plasmids described above using the CaPQ4 method, which involves a mixture of Tris:EDTA, 2M CaCh, 2×HBS, and the four DNA plasmids. Day 3 after transfection, the supernatant containing secreted lentiviral vectors is collected, purified and concentrated. After the supernatant is removed from the CFs, End-of-Production Cells are collected from each CF. Cells are trypsinized from each factory and collected by centrifugation. Cells are resuspended in freezing medium and cryopreserved. These cells are later used for replication-competent lentivirus (RCL) testing.

To purify and formulate vectors crude, supernatant is clarified by membrane filtration to remove the cell debris. The host cell DNA and residual plasmid DNA are degraded by endonuclease digestion (Benzonase®). The viral supernatant is clarified of cellular debris using a 0.45 μm filter. The clarified supernatant is collected into a pre-weighed container into which the Benzonase® is added (final concentration 50 U/mL). The endonuclease digestion for residual plasmid DNA and host genomic DNA is performed at 37° C. for 6 h. The initial tangential flow ultrafiltration (TFF) concentration of the endonuclease-treated supernatant is used to remove residual low molecular weight components from the crude supernatant, while concentrating the virus ˜20 fold. The clarified endonuclease-treated viral supernatant is circulated through a hollow fiber cartridge with a NMWCO of 500 kD at a flow rate designed to maintain the shear rate at ˜4,000 sec⁻¹ or less, while maximizing the flux rate. Diafiltration of the nuclease-treated supernatant is initiated during the concentration process to sustain the cartridge performance. An 80% permeate replacement rate is established, using 4% lactose in PBS as the diafiltration buffer. The viral supernatant is brought to the target volume, representing a 20-fold concentration of the crude supernatant, and the diafiltration is continued for 4 additional exchange volumes, with the permeate replacement rate at 100%.

Further concentration of the viral product is accomplished by using a high speed centrifugation technique. Each sub-batch of the lentivirus is pelleted using a Sorvall RC-26 plus centrifuge at 6000 RPM (6,088 RCF) at 6° C. for 16-20 h. The viral pellet from each sub-batch is then reconstituted in a 50 mL volume with 4% lactose in PBS. The reconstituted pellet in this buffer represents the final formulation for the virus preparation. The entire vector concentration process results in a 200-fold volume reduction, approximately. Following the completion of all of the sub-batches, the material is then placed at −80° C., while samples from each sub-batch are tested for sterility. Following confirmation of sample sterility, the sub-batches are rapidly thawed at 37° C. with frequent agitation. The material is then pooled and manually aliquoted in the Class II Type A/B3 biosafety cabinet. A fill configuration of 1 mL of the concentrated lentivirus in sterile USP class 6, externally threaded 0-ring cryovials is used.

To ensure the purity of the lentiviral vector preparation, it is tested for residual host DNA contaminants, and the transfer of residual host and plasmid DNA. Among other tests, vector identity is evaluated by RT-PCR to ensure that the correct vector is present.

### Example 4: Preparation of T Cells Suitable for Use in ACT

If T_{CM} are to be used to express the CAR, suitable patient cells can be prepared as follows. First, T lymphocytes are obtained from a patient by leukopheresis, and the appropriate allogenic or autologous T cell subset, for example, Central Memory T cells (T_{CM}), are genetically altered to express the CAR, then administered back to the patient by any clinically acceptable means, to achieve anti-cancer therapy.

Suitable TcM can be generated as follow. Apheresis products obtained from consented research participants are ficolled, washed and incubated overnight. Cells are then depleted of monocyte, regulatory T cell and naïve T cell populations using GMP grade anti-CD14, anti-CD25 and anti-CD45RA reagents (Miltenyi Biotec) and the CliniMACS™ separation device. Following depletion, negative fraction cells are enriched for CD62L+ T_{CM} cells using DREG56-biotin (COH clinical grade) and anti-biotin microbeads (Miltenyi Biotec) on the CliniMACS™ separation device.

Following enrichment, TCM cells are formulated in complete X-Vivo15 plus 50 IU/mL IL-2 and 0.5 ng/ml IL-15 and transferred to a Teflon cell culture bag, where they are stimulated with Dynal ClinEx™ Vivo CD3/CD28 beads. Up to five days after stimulation, cells are transduced with lentiviral vector expressing the desired CAR at a multiplicity of infection (MOI) of 1.0 to 0.3. Cultures are maintained for up to 42 days with addition of complete X-Vivo15 and IL-2 and IL-15 cytokine as required for cell expansion (keeping cell density between 3×10⁵ and 2×10⁶ viable cells/mL, and cytokine supplementation every Monday, Wednesday and Friday of culture). Cells typically expand to approximately 10⁹ cells under these conditions within 21 days. At the end of the culture period cells are harvested, washed twice and formulated in clinical grade cryopreservation medium (Cryostore CS5, BioLife Solutions).

On the day(s) of T cell infusion, the cryopreserved and released product is thawed, washed and formulated for re-infusion. The cryopreserved vials containing the released cell product are removed from liquid nitrogen storage, thawed, cooled and washed with a PBS/2% human serum albumin (HSA) Wash Buffer. After centrifugation, the supernatant is removed and the cells resuspended in a Preservative-Free Normal Saline (PFNS)/2% HSA infusion diluent. Samples are removed for quality control testing.

### Example 5: Expression of CAR Targeted to HER2

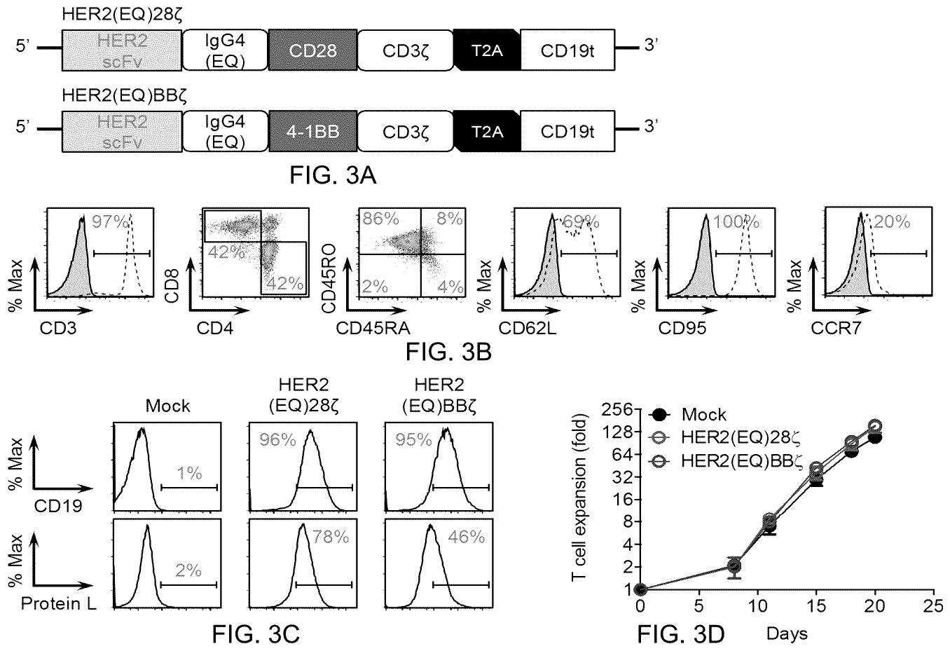

FIG. 3A is a schematic diagram of two the HER2-specific CAR constructs depicted in FIG. 1 and FIG. 2. In HER2(EQ)28ξ the scFv is tethered to the membrane by a modified IgG4 Fc linker (double mutant, L235E; N297Q), containing a CD28 transmembrane domain, an intracellular CD28 co-stimulatory domain and a cytolytic CD3ξ domain. The T2A skip sequence separates the CAR from a truncated CD19 (CD19t) protein employed for cell tracking. HER2(EQ)BBξ is similar except that the costimulatory domain is 4-1BB rather than CD28 and the transmembrane domain is a CD8 transmembrane domain rather than a CD28 transmembrane domain. Human central memory (TCM) cells were transfected with a lentiviral vector expressing either HER2(EQ)28ξ or HER2(EQ)BBξ. FIG. 3B depicts representative FACS data of human TCM surface phenotype. FIG. 3C depicts the results of assays for CD 19 and Protein L expression in TCM transfected with a lentiviral vector expressing either HER2(EQ)28ξ or HER2(EQ)BBξ. As can be seen from these results, transfection efficiency as assessed by CD19 expression was similar for both CAR. However, Protein L expression was lower for HER2(EQ)BBξ than for HER2(EQ)28ξ suggesting that the HER2(EQ)BBξ CAR is less stable that the HER2(EQ)BBξ. Analysis of cell expansion (FIG. 3D) shows that neither CAR interferes with T cell expansion.

### Example 6: In Vitro Characterization of HER2-CAR T Cells Against Various Breast Cancer Cell Lines

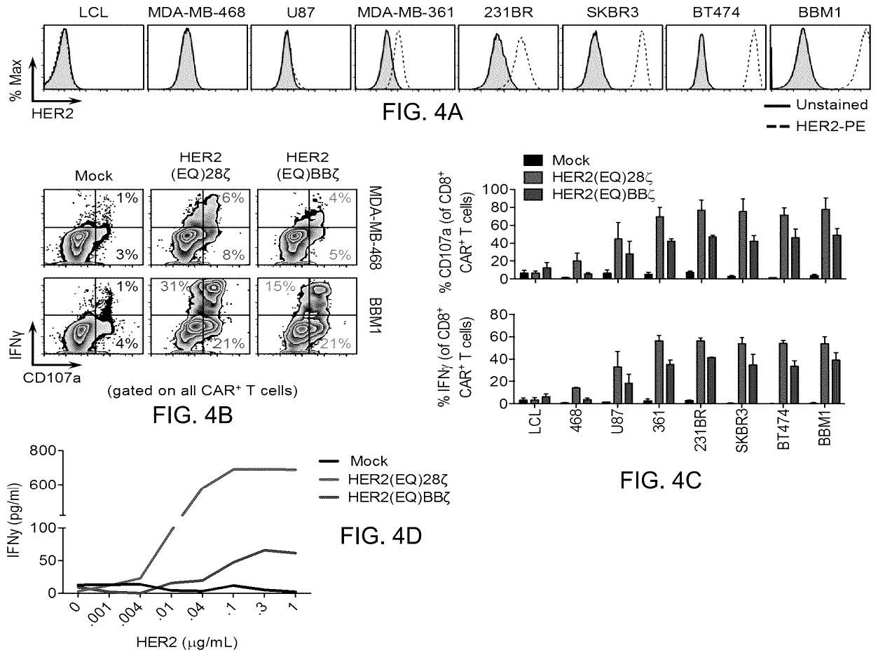

A variety of breast cancer cell lines, including, HER2-negative lines (LCL lymphoma, MDA-MB-468, U87 glioma), low-HER2 expressing lines (MDA-MB-361, 231BR) and high-HER2 expressing lines (SKBR3, BT474, BBM1) were used to characterize HER2(EQ)28 and HER2(EQ)BBξ. FIG. 4A depicts the HER2 expression level of each of these lines. Flow cytometry (gated on CAR+ T cells) was used to characterize CD107a degranulation and IFNy production in Mock (untransduced), HER2(EQ)28ξ or HER2(EQ)BBξ CAR T cells following a 5 hr co-culture with either MDA-MB-361 tumor cells (low HER2 expressing) or BBM1 tumor cells (high HER2 expressing). The results of this analysis are presented in FIG. 4B. Similar studies were conducted with the other breast cancer cells lines, and the results are summarized in FIG. 4C. Production of IFNγ production by HER2-CAR T cells following a 24 hr culture with recombinant HER2 protein or tumor targets was measured by ELISA and the results of this analysis are shown in FIG. 4D.

### Example 7: In Vitro Anti-Tumor Activity

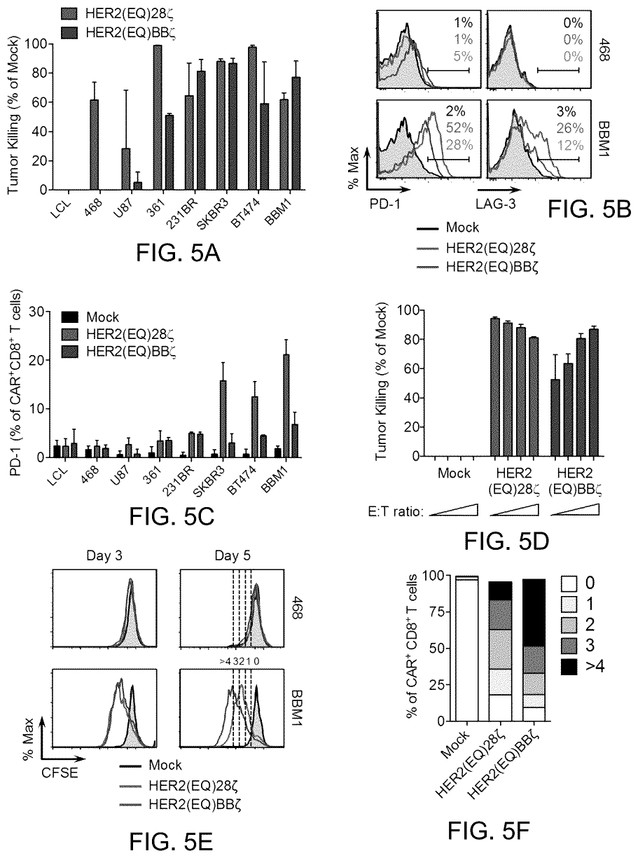

Flow cytometry was used to assess tumor cell killing following a 72 h co-culture of Mock (untransduced), HER2(EQ)28ξ or HER2(EQ)BBξ CAR T cells with tumor targets. The results of this analysis are presented in FIG. 5A. PD-1 and LAG-3 induction in total CAR T cells after a 72 h co-culture with HER2-negative MDA-MB-468 or HER2-positive BBM1 cells was measured, and the results of this analysis are presented in FIG. 5B. PD-1 induction in CD8+ CAR T cells following a 72 h co-culture with tumor targets that are HER2-negative (LCL lymphoma, MDA-MB-468, U87 glioma), low-HER2 expressing (MDA-MB-361, 231BR) or high-HER2 expressing (SKBR3, BT474, BBM 1) was measured, and the results of this analysis are presented in FIG. 5C. These studies suggest that HER2(EQ)BBξ causes lower PD-1 induction that does HER2(EQ)28ξ. Tumor cell killing with Effector:Tumor (E:T) ratio ranging from 0.25:1 to 2:1 was measured for both HER2(EQ)28ξ or HER2(EQ)BBξ CAR T cells. The results of this analysis are presented in FIG. 5D, which shows that both HER2(EQ)28ξ and HER2(EQ)BBξ are effective in tumor cell killing in vitro. CFSE proliferation of HER2-CAR T cells following a 72 h co-culture with MDA-MB-468 or BBMI cells was measured by flow cytometry. The results of this analysis are presented in FIG. 5E, which shows that HER2(EQ)BBξ CAR T cells proliferate more than HER2(EQ)28ξ CAR T cells.

### Example 8: In Vivo Anti-Tumor Activity

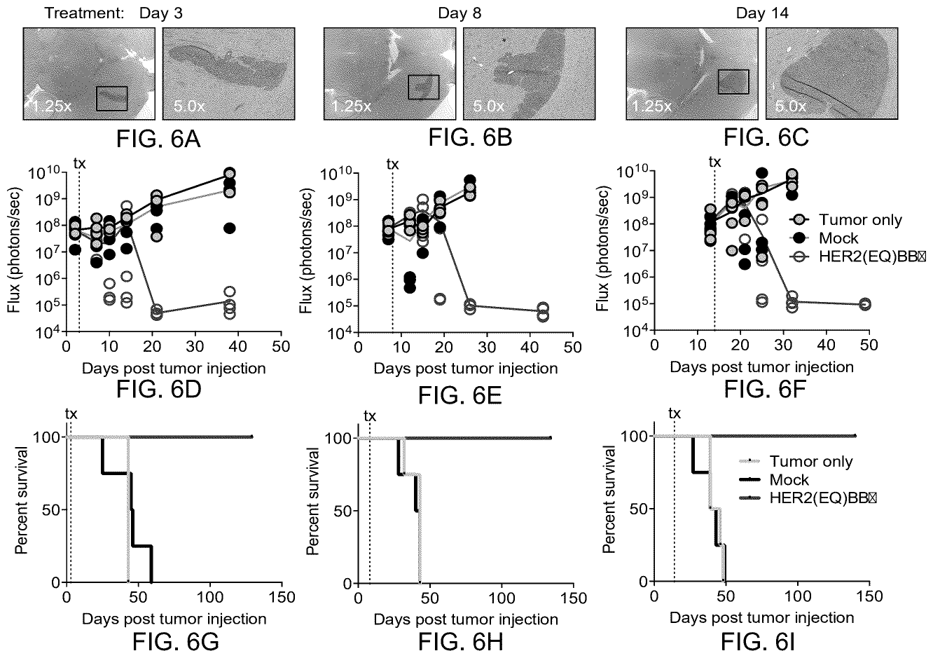

The activity of intratumorally delivered HER2 CAR T cells was assessed in a patient-derived breast-to-brain metastasis model. FIGS. 6A-6C are H&E staining of tumors. Mice were treating by injection directly into the tumor with Mock (untransduced) or HER2(EQ)BBξ CAR T cells. FIGS. 6D-6F depict the results of optical imaging of the tumors and FIGS. 6G-6I are Kaplan-Meier survival curves for mice treated locally with either at day 3, 8 or 14 post tumor injection. These studies show that HER2(EQ)BBξ CAR T cells have potent anti-tumor efficacy in vivo when injected directly into the tumor.

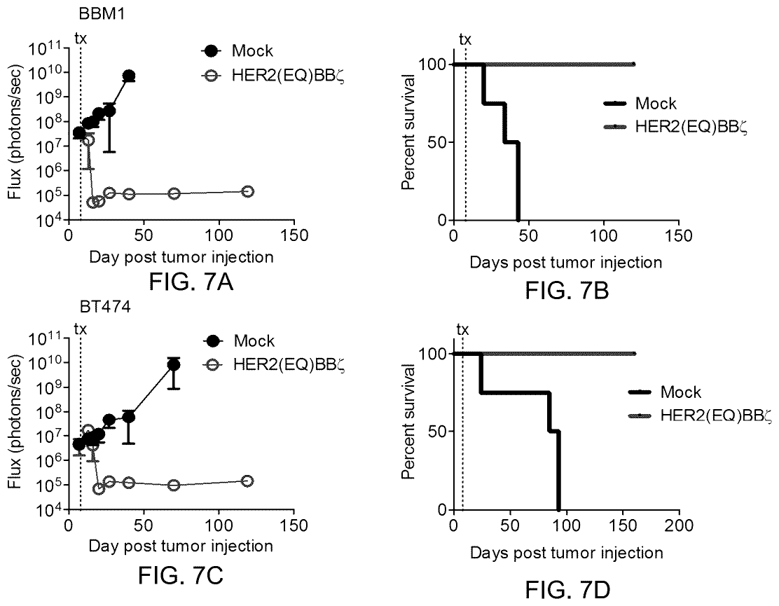

To assess anti-tumor efficacy in human xenograft models of breast-to-brain metastasis, BBM1 cells (0.2M) or BT474 (0.15M) were intracranially injected in NSG mice. At day 8 post tumor injection, HER2(EQ)28ξ or HER2(EQ)BBξ, or Mock (untransduced) T cells (IM) were injected intratumorally. BBMI (FIG. 7A) and BT474 (FIG. 7B) tumors were monitored by luciferase-based optical imaging. Kaplan Meier curves are presented in FIG. 7C and FIG. 7D.

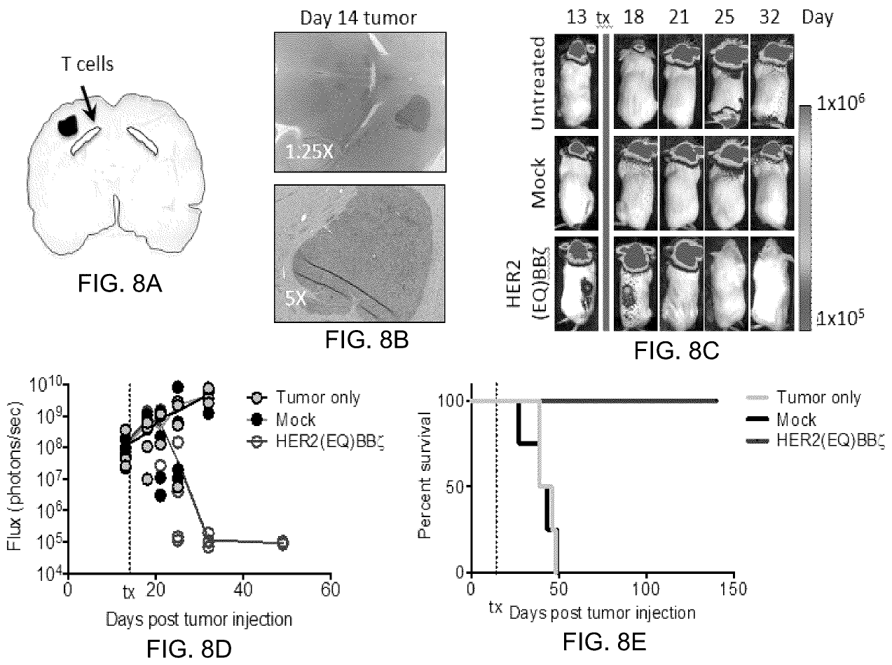

A human patient-derived orthotopic xenograft model of breast-to-brain metastasis was also used to assess HER2(EQ)28ξ and HER2(EQ)BBξ CAR T cells. FIG. 8A illustrates the region of tumor implantation by stereotactic injection of BBM1 cells (0.2M), and intraventricular T cell delivery. Staining of tumors is depicted in FIG. 8B. At day 14 post tumor injection, HER2(EQ)28ξ, HER2(EQ)BBξ, or Mock (untransduced) T cells (0.5M) were injected intratumorally. Tumor growth was monitored by luciferase-based optical imaging. FIG. 8C presents the flux averages for each treatment group, and FIG. 8D presents the Kaplan Meier survival curve for each treatment group.

### Example 9: Additional CAR Targeted to HER2

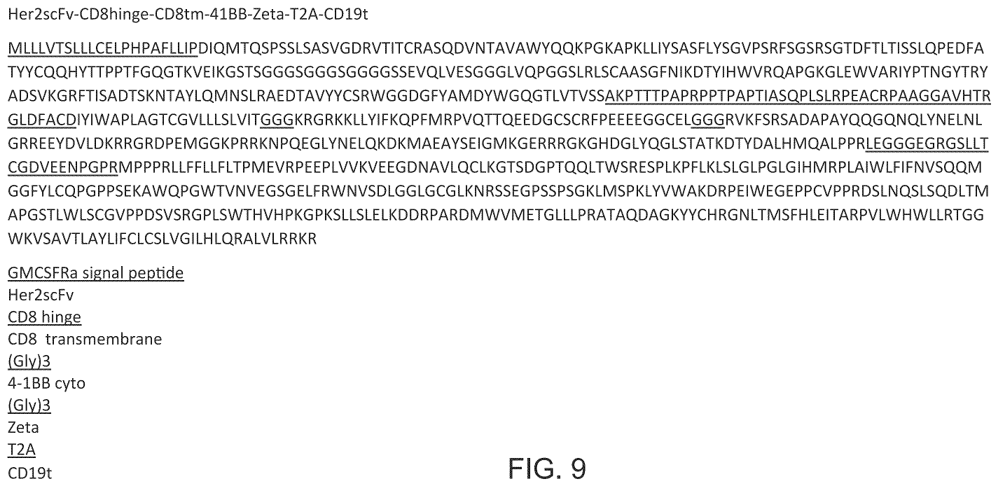

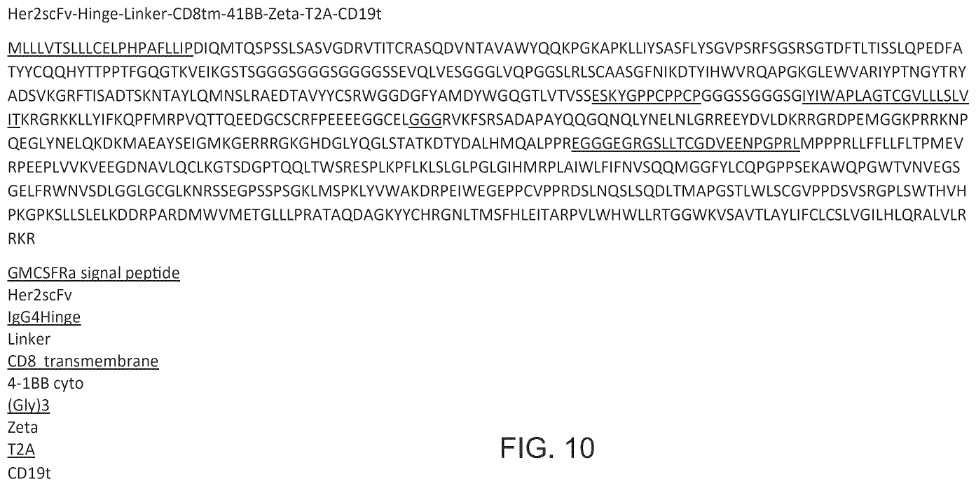

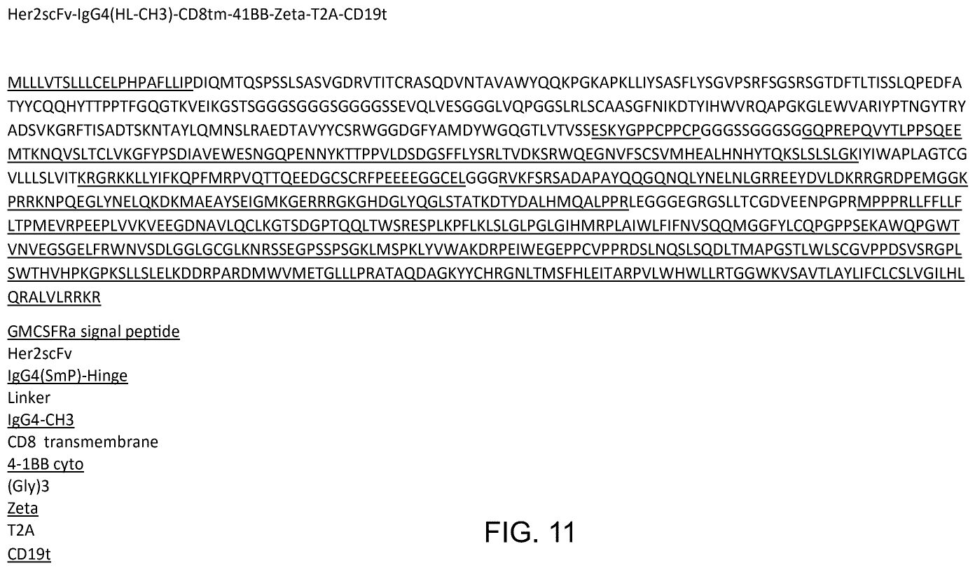

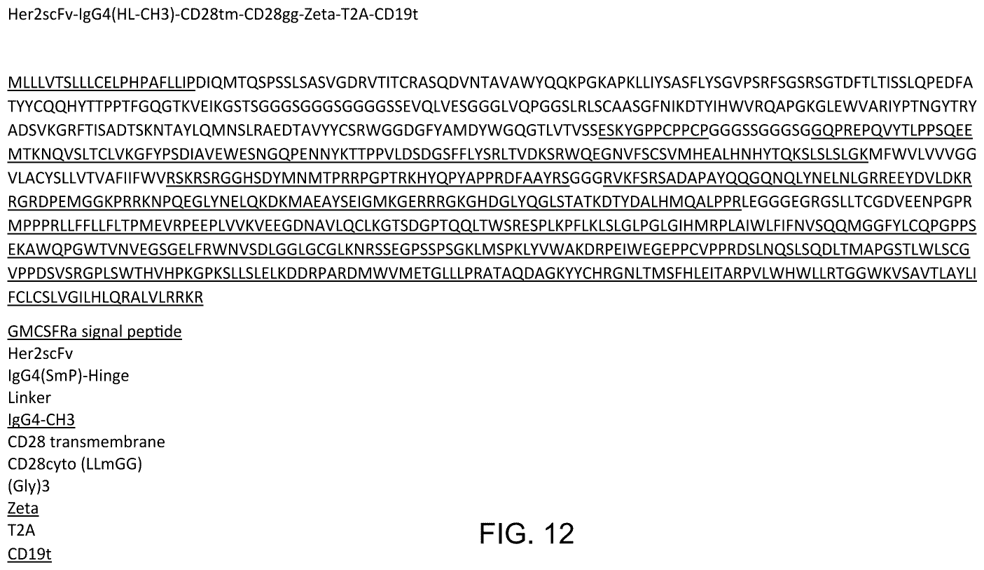

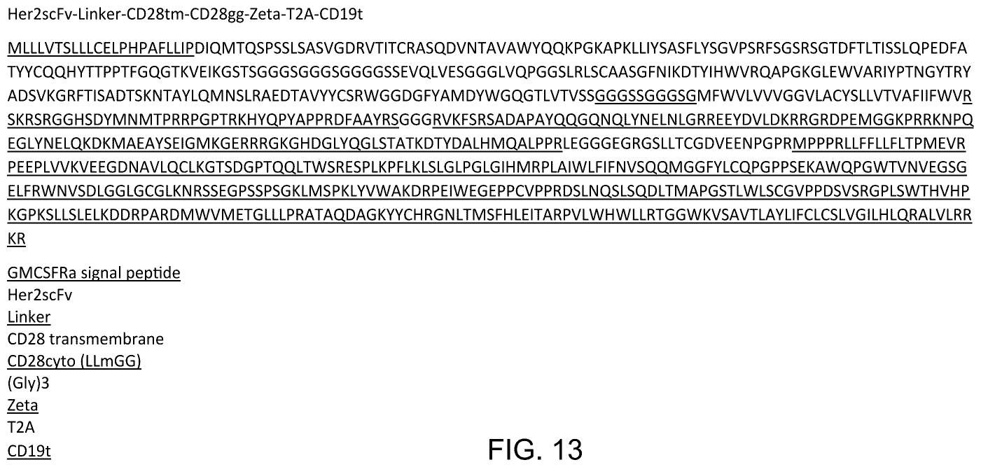

FIGS. 9-14 depict the amino acid sequences of a various CAR having different linkers. Specifically, the CAR differ in the sequence and length of the portion between the HER2 targeted scFv and the transmembrane domain. The transmembrane domain is CD8, CD28 or CD28gg. The co-stimulatory domain is 4-1BB or CD28. All have a CD3ζ stimulatory domain. In each case a T2A skip sequence separates the CAR from a truncated CD19 (CD19t) protein employed for cell tracking.

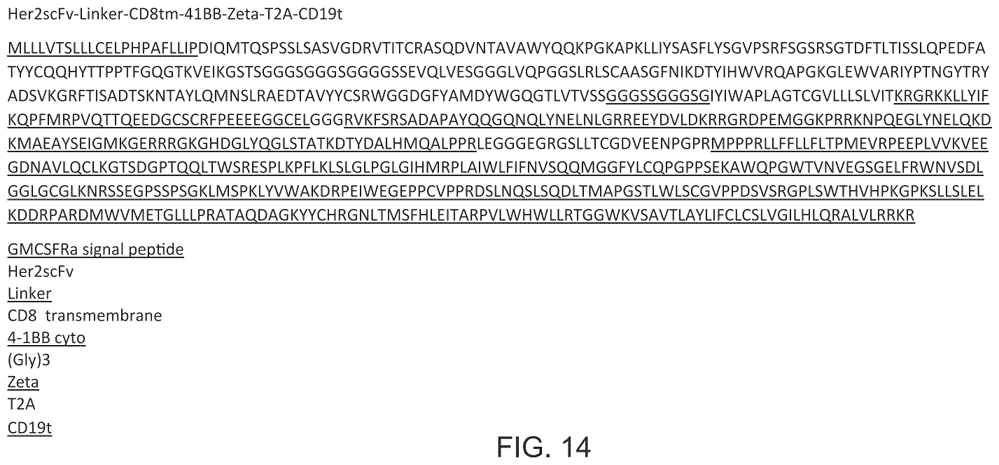

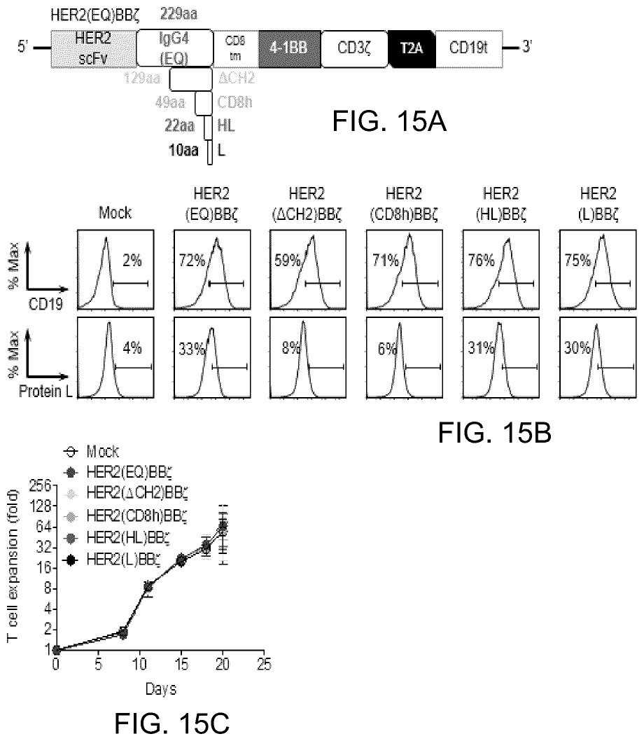

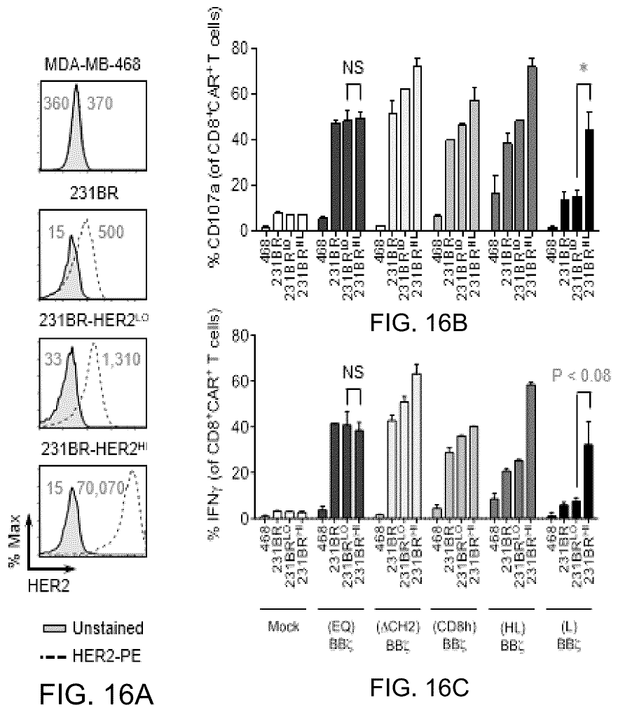

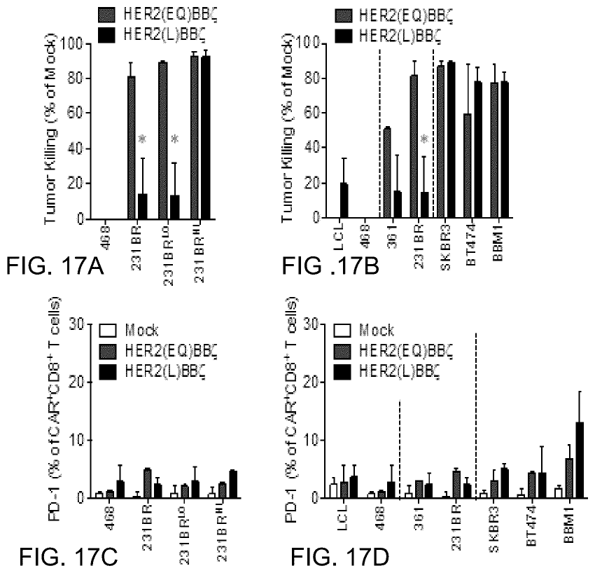

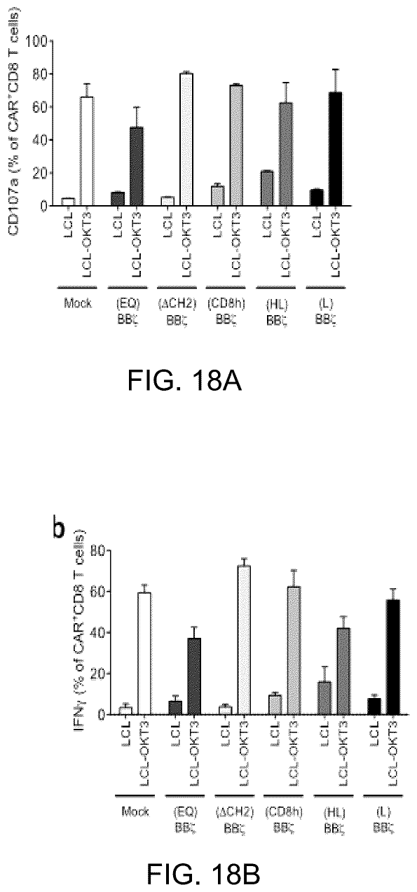

FIG. 15A schematically depicts various HER2 CAR that are identical except for the sequence and length of the portion between the HER2 scFv and the CD8 transmembrane domain. All include a 4-1BB co-stimulatory domain followed by a CD35 stimulatory domain. FIG. 15B depicts the results of assays for CD19 and Protein L expression in T_{CM} transfected with a lentiviral vector expressing the indicated CAR. As can be seen from these results, transfection efficiency as assessed by CD19 expression was similar for both CAR. However, Protein L expression was lower for HER2(EQ)BBζ than for HER2(EQ)28ζ suggesting that the HER2(EQ)BBζ CAR is less stable that the HER2(EQ)BBζ. Analysis of cell expansion (FIG. 15C) shows that none of the CAR interfere with T cell expansion. FIGS. 16-18 show the results of additional studies showing that a CAR with a very short spacer (FIG. 14) is relatively selective for CAR expressing high levels of HER2. Such CAR may be useful in treating HER2 expressing cancers where it is desirable to spare cells expressing a lower level of HER2 than the cancerous cells.

1. A nucleic acid molecule encoding a chimeric antigen receptor, wherein the chimeric antigen receptor comprises: a HER2 targeting sequence; a transmembrane domain selected from: a CD4 transmembrane domain or variant thereof having 1-5 amino acid modifications, a CD8 transmembrane domain or variant thereof having 1-5 amino acid modifications, a CD28 transmembrane domain or a variant thereof having 1-5 amino acid modifications, and a CD3ξ transmembrane domain or a variant thereof having 1-5 amino acid modifications; a costimulatory domain selected from a CD28 costimulatory domain or a variant thereof having 1-5 amino acid modifications and a 4-IBB costimulatory domain or a variant thereof having 1-5 amino acid modifications; and CD3ξ signaling domain of a variant thereof having 1-5 amino acid modifications.

2. The nucleic acid molecule of claim 1 wherein the HER2 targeting domain is a HER2 scFv.

3. The nucleic acid molecule of claim 1 wherein the HER2 scFv comprising the amino acid sequence:
DIQMTQSPSSLSASVGDRVTITCRASQDVNTAVAWYQQKPGKAPKLLIYSASFLYSGVPS RFSGSRSGTDFTLTISSLQPEDFATYYCQQHYTTPPTFGQGTKVEIKGSTSGGGSGGGSG GGGSSEVOL VESGGGL VQPGGSLRLSCAASGFNIKDTYIHWVRQAPGKGLEWVARIYP TNGYTRYADSVKGRFTISADTSKNTAYLQMNSLRAEDTAVYYCSRWGGDGFYAMDY WGQGTLVTVSS or a variant thereof having 1 to 5 amino acid modifications.

4. (canceled)

5. The nucleic acid molecule of claim 1 comprising a spacer region located between the HER2 targeting domain and the transmembrane domain.

6. (canceled)

7. The nucleic acid molecule of claim 5 wherein the spacer region comprises an amino acid sequence selected from the group consisting of SEQ ID NOs: 2-12 or a variant thereof having 1-5 amino acid modifications.

8. (canceled)

9. (canceled)

10. The nucleic acid molecule of claim 1 wherein the costimulatory domain is a 4-1 BB costimulatory domain comprising the amino acid sequence of SEQ ID NO:24 or a variant thereof having 1-5 amino acid modifications.

11. (canceled)

12. The nucleic acid molecule of claim 1 wherein a linker of 3 to 15 amino acids is located between the costimulatory domain or the variant thereof and the CD3ξ signaling domain or variant thereof.

13. The nucleic acid molecule of claim 1 wherein the nucleic acid molecule expresses a polypeptide comprising an amino acid sequence selected from SEQ ID NO: 26 and 27 or a variant thereof having 1-5 amino acid modifications.

14. The nucleic acid molecule of claim 1 wherein the chimeric antigen receptor comprises a 4-1 BB costimulatory domain and a spacer region comprising the amino acid sequence of any of SEQ ID NOs: 2-12 or a variant thereof having 1-5 amino acid modifications.

15. The nucleic acid molecule of claim 1 wherein the chimeric antigen receptor comprises the amino acid sequence selected from SEQ ID NOs: 26 and 27.

16. (canceled)

17. (canceled)

18. A population of human T cells transduced by a vector comprising the nucleic acid molecule of claim 1.

19. (canceled)

20. (canceled)

21. A method of treating a HER2 expressing brain cancer in a patient comprising administering a population of autologous or allogeneic human T cells transduced by a vector comprising an expression cassette encoding a chimeric antigen receptor, wherein chimeric antigen receptor comprises an amino acid sequence selected from SEQ ID NOs: 26 and 27 or a variant thereof having 1-5 amino acid modifications.

22. The method of claim 21 wherein the population of human T cells comprise CD62L+ memory T cells.

23. The method claim 21 wherein the cancer is a breast to brain metastasis.

24. The method of claim 21 wherein the transduced human T cells where prepared by a method comprising obtaining T cells from the patient, treating the T cells to isolate central memory T cells, and transducing at least a portion of the central memory cells to with a viral vector comprising an expression cassette encoding a chimeric antigen receptor, wherein chimeric antigen receptor comprises an amino acid sequence selected from SEQ ID NOs: 26 and 27 or a variant thereof having 1-5 amino acid modifications.

25. The method of claim 21 wherein the T cells are administered intratumorally.

26. The method of claim 21 wherein the T cells are administered intraventricularly.

27. The method of claim 21 wherein the T cells are administered intraventricularly adjacent to a tumor.

28. (canceled)

29. A T cell expressing a polypeptide comprising an amino acid sequence that is identical to an amino acid sequence selected from SEQ ID NOs: 26 and 27 or a variant thereof having 1-5 amino acid modifications.

30. The nucleic acid molecule of claim 1 wherein the nucleic acid molecule encodes a polypeptide comprising the amino acid sequence of any of SEQ ID NOs: 26-41.

31. (canceled)

32. (canceled)


SEQUENCE LISTING (42 sequences)

SEQ ID NO: 1 - protein, 245 aa, Homo sapiens
DIQMTQSPSSLSASVGDRVTITCRASQDVNTAVAWYQQKPGKAPKLLIYSASFLYSGVPSRFSGSRSGTDFTLTISSLQPEDFATYYCQQHYTTPPTFGQGTKVEIKGSTSGGGSGGGSGGGGSSEVQLVESGGGLVQPGGSLRLSCAASGFNIKDTYIHWVRQAPGKGLEWVARIYPTNGYTRYADSVKGRFTISADTSKNTAYLQMNSLRAEDTAVYYCSRWGGDGFYAMDYWGQGTLVTVSS

SEQ ID NO: 2 - protein, 10 aa, synthetic construct
GGGSSGGGSG

SEQ ID NO: 3 - protein, 12 aa, synthetic construct
ESKYGPPCPPCP

SEQ ID NO: 4 - protein, 12 aa, Homo sapiens
ESKYGPPCPSCP

SEQ ID NO: 5 - protein, 22 aa, synthetic construct
ESKYGPPCPPCPGGGSSGGGSG

SEQ ID NO: 6 - protein, 39 aa, Homo sapiens
IEVMYPPPYLDNEKSNGTIIHVKGKHLCPSPLFPGPSKP

SEQ ID NO: 7 - protein, 48 aa, Homo sapiens
AKPTTTPAPRPPTPAPTIASQPLSLRPEACRPAAGGAVHTRGLDFACD

SEQ ID NO: 8 - protein, 45 aa, Homo sapiens
TTTPAPRPPTPAPTIASQPLSLRPEACRPAAGGAVHTRGLDFACD

SEQ ID NO: 9 - protein, 129 aa, synthetic construct
ESKYGPPCPPCPGGGSSGGGSGGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 10 - protein, 228 aa, synthetic construct
ESKYGPPCPSCPAPEFEGGPSVFLFPPKPKDTLMISRTPEVTCVVVDVSQEDPEVQFNWYVDGVEVHNAKTKPREEQFSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 11 - protein, 229 aa, synthetic construct
ESKYGPPCPPCPAPEFEGGPSVFLFPPKPKDTLMISRTPEVTCVVVDVSQEDPEVQFNWYVDGVEVHNAKTKPREEQFQSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 12 - protein, 107 aa, Homo sapiens
GQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 13 - protein, 21 aa, Homo sapiens
LCYLLDGILFIYGVILTALFL

SEQ ID NO: 14 - protein, 27 aa, Homo sapiens
FWVLVVVGGVLACYSLLVTVAFIIFWV

SEQ ID NO: 15 - protein, 28 aa, Homo sapiens
MFWVLVVVGGVLACYSLLVTVAFIIFWV

SEQ ID NO: 16 - protein, 22 aa, Homo sapiens
MALIVLGGVAGLLLFIGLGIFF

SEQ ID NO: 17 - protein, 21 aa, Homo sapiens
IYIWAPLAGTCGVLLLSLVIT

SEQ ID NO: 18 - protein, 23 aa, Homo sapiens
IYIWAPLAGTCGVLLLSLVITLY

SEQ ID NO: 19 - protein, 24 aa, Homo sapiens
IYIWAPLAGTCGVLLLSLVITLYC

SEQ ID NO: 20 - protein, 28 aa, Homo sapiens
IISFFLALTSTALLLFLLFFLTLRFSVV

SEQ ID NO: 21 - protein, 112 aa, Homo sapiens
RVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 22 - protein, 41 aa, Homo sapiens
RSKRSRLLHSDYMNMTPRRPGPTRKHYQPYAPPRDFAAYRS

SEQ ID NO: 23 - protein, 41 aa, synthetic construct
RSKRSRGGHSDYMNMTPRRPGPTRKHYQPYAPPRDFAAYRS

SEQ ID NO: 24 - protein, 42 aa, Homo sapiens
KRGRKKLLYIFKQPFMRPVQTTQEEDGCSCRFPEEEEGGCEL

SEQ ID NO: 25 - protein, 42 aa, Homo sapiens
ALYLLRRDQRLPPDAHKPPGGGSFRTPIQEEQADAHSTLAKI

SEQ ID NO: 26 - protein, 1027 aa, synthetic construct
MLLLVTSLLLCELPHPAFLLIPDIQMTQSPSSLSASVGDRVTITCRASQDVNTAVAWYQQKPGKAPKLLIYSASFLYSGVPSRFSGSRSGTDFTLTISSLQPEDFATYYCQQHYTTPPTFGQGTKVEIKGSTSGGGSGGGSGGGGSSEVQLVESGGGLVQPGGSLRLSCAASGFNIKDTYIHWVRQAPGKGLEWVARIYPTNGYTRYADSVKGRFTISADTSKNTAYLQMNSLRAEDTAVYYCSRWGGDGFYAMDYWGQGTLVTVSSESKYGPPCPPCPAPEFEGGPSVFLFPPKPKDTLMISRTPEVTCVVVDVSQEDPEVQFNWYVDGVEVHNAKTKPREEQFQSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGKMFWVLVVVGGVLACYSLLVTVAFIIFWVRSKRSRGGHSDYMNMTPRRPGPTRKHYQPYAPPRDFAAYRSGGGRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPRLEGGGEGRGSLLTCGDVEENPGPRMPPPRLLFFLLFLTPMEVRPEEPLVVKVEEGDNAVLQCLKGTSDGPTQQLTWSRESPLKPFLKLSLGLPGLGIHMRPLAIWLFIFNVSQQMGGFYLCQPGPPSEKAWQPGWTVNVEGSGELFRWNVSDLGGLGCGLKNRSSEGPSSPSGKLMSPKLYVWAKDRPEIWEGEPPCVPPRDSLNQSLSQDLTMAPGSTLWLSCGVPPDSVSRGPLSWTHVHPKGPKSLLSLELKDDRPARDMWVMETGLLLPRATAQDAGKYYCHRGNLTMSFHLEITARPVLWHWLLRTGGWKVSAVTLAYLIFCLCSLVGILHLQRALVLRRKR

SEQ ID NO: 27 - protein, 1021 aa, synthetic construct
MLLLVTSLLLCELPHPAFLLIPDIQMTQSPSSLSASVGDRVTITCRASQDVNTAVAWYQQKPGKAPKLLIYSASFLYSGVPSRFSGSRSGTDFTLTISSLQPEDFATYYCQQHYTTPPTFGQGTKVEIKGSTSGGGSGGGSGGGGSSEVQLVESGGGLVQPGGSLRLSCAASGFNIKDTYIHWVRQAPGKGLEWVARIYPTNGYTRYADSVKGRFTISADTSKNTAYLQMNSLRAEDTAVYYCSRWGGDGFYAMDYWGQGTLVTVSSESKYGPPCPPCPAPEFEGGPSVFLFPPKPKDTLMISRTPEVTCVVVDVSQEDPEVQFNWYVDGVEVHNAKTKPREEQFQSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGKIYIWAPLAGTCGVLLLSLVITKRGRKKLLYIFKQPFMRPVQTTQEEDGCSCRFPEEEEGGCELGGGRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPRLEGGGEGRGSLLTCGDVEENPGPRMPPPRLLFFLLFLTPMEVRPEEPLVVKVEEGDNAVLQCLKGTSDGPTQQLTWSRESPLKPFLKLSLGLPGLGIHMRPLAIWLFIFNVSQQMGGFYLCQPGPPSEKAWQPGWTVNVEGSGELFRWNVSDLGGLGCGLKNRSSEGPSSPSGKLMSPKLYVWAKDRPEIWEGEPPCVPPRDSLNQSLSQDLTMAPGSTLWLSCGVPPDSVSRGPLSWTHVHPKGPKSLLSLELKDDRPARDMWVMETGLLLPRATAQDAGKYYCHRGNLTMSFHLEITARPVLWHWLLRTGGWKVSAVTLAYLIFCLCSLVGILHLQRALVLRRKR

SEQ ID NO: 28 - protein, 843 aa, synthetic construct
MLLLVTSLLLCELPHPAFLLIPDIQMTQSPSSLSASVGDRVTITCRASQDVNTAVAWYQQKPGKAPKLLIYSASFLYSGVPSRFSGSRSGTDFTLTISSLQPEDFATYYCQQHYTTPPTFGQGTKVEIKGSTSGGGSGGGSGGGGSSEVQLVESGGGLVQPGGSLRLSCAASGFNIKDTYIHWVRQAPGKGLEWVARIYPTNGYTRYADSVKGRFTISADTSKNTAYLQMNSLRAEDTAVYYCSRWGGDGFYAMDYWGQGTLVTVSSAKPTTTPAPRPPTPAPTIASQPLSLRPEACRPAAGGAVHTRGLDFACDIYIWAPLAGTCGVLLLSLVITGGGKRGRKKLLYIFKQPFMRPVQTTQEEDGCSCRFPEEEEGGCELGGGRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPRLEGGGEGRGSLLTCGDVEENPGPRMPPPRLLFFLLFLTPMEVRPEEPLVVKVEEGDNAVLQCLKGTSDGPTQQLTWSRESPLKPFLKLSLGLPGLGIHMRPLAIWLFIFNVSQQMGGFYLCQPGPPSEKAWQPGWTVNVEGSGELFRWNVSDLGGLGCGLKNRSSEGPSSPSGKLMSPKLYVWAKDRPEIWEGEPPCVPPRDSLNQSLSQDLTMAPGSTLWLSCGVPPDSVSRGPLSWTHVHPKGPKSLLSLELKDDRPARDMWVMETGLLLPRATAQDAGKYYCHRGNLTMSFHLEITARPVLWHWLLRTGGWKVSAVTLAYLIFCLCSLVGILHLQRALVLRRKR

SEQ ID NO: 29 - protein, 814 aa, synthetic construct
MLLLVTSLLLCELPHPAFLLIPDIQMTQSPSSLSASVGDRVTITCRASQDVNTAVAWYQQKPGKAPKLLIYSASFLYSGVPSRFSGSRSGTDFTLTISSLQPEDFATYYCQQHYTTPPTFGQGTKVEIKGSTSGGGSGGGSGGGGSSEVQLVESGGGLVQPGGSLRLSCAASGFNIKDTYIHWVRQAPGKGLEWVARIYPTNGYTRYADSVKGRFTISADTSKNTAYLQMNSLRAEDTAVYYCSRWGGDGFYAMDYWGQGTLVTVSSESKYGPPCPPCPGGGSSGGGSGIYIWAPLAGTCGVLLLSLVITKRGRKKLLYIFKQPFMRPVQTTQEEDGCSCRFPEEEEGGCELGGGRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPREGGGEGRGSLLTCGDVEENPGPRLMPPPRLLFFLLFLTPMEVRPEEPLVVKVEEGDNAVLQCLKGTSDGPTQQLTWSRESPLKPFLKLSLGLPGLGIHMRPLAIWLFIFNVSQQMGGFYLCQPGPPSEKAWQPGWTVNVEGSGELFRWNVSDLGGLGCGLKNRSSEGPSSPSGKLMSPKLYVWAKDRPEIWEGEPPCVPPRDSLNQSLSQDLTMAPGSTLWLSCGVPPDSVSRGPLSWTHVHPKGPKSLLSLELKDDRPARDMWVMETGLLLPRATAQDAGKYYCHRGNLTMSFHLEITARPVLWHWLLRTGGWKVSAVTLAYLIFCLCSLVGILHLQRALVLRRKR

SEQ ID NO: 30 - protein, 921 aa, synthetic construct
MLLLVTSLLLCELPHPAFLLIPDIQMTQSPSSLSASVGDRVTITCRASQDVNTAVAWYQQKPGKAPKLLIYSASFLYSGVPSRFSGSRSGTDFTLTISSLQPEDFATYYCQQHYTTPPTFGQGTKVEIKGSTSGGGSGGGSGGGGSSEVQLVESGGGLVQPGGSLRLSCAASGFNIKDTYIHWVRQAPGKGLEWVARIYPTNGYTRYADSVKGRFTISADTSKNTAYLQMNSLRAEDTAVYYCSRWGGDGFYAMDYWGQGTLVTVSSESKYGPPCPPCPGGGSSGGGSGGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGKIYIWAPLAGTCGVLLLSLVITKRGRKKLLYIFKQPFMRPVQTTQEEDGCSCRFPEEEEGGCELGGGRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPRLEGGGEGRGSLLTCGDVEENPGPRMPPPRLLFFLLFLTPMEVRPEEPLVVKVEEGDNAVLQCLKGTSDGPTQQLTWSRESPLKPFLKLSLGLPGLGIHMRPLAIWLFIFNVSQQMGGFYLCQPGPPSEKAWQPGWTVNVEGSGELFRWNVSDLGGLGCGLKNRSSEGPSSPSGKLMSPKLYVWAKDRPEIWEGEPPCVPPRDSLNQSLSQDLTMAPGSTLWLSCGVPPDSVSRGPLSWTHVHPKGPKSLLSLELKDDRPARDMWVMETGLLLPRATAQDAGKYYCHRGNLTMSFHLEITARPVLWHWLLRTGGWKVSAVTLAYLIFCLCSLVGILHLQRALVLRRKR

SEQ ID NO: 31 - protein, 927 aa, synthetic construct
MLLLVTSLLLCELPHPAFLLIPDIQMTQSPSSLSASVGDRVTITCRASQDVNTAVAWYQQKPGKAPKLLIYSASFLYSGVPSRFSGSRSGTDFTLTISSLQPEDFATYYCQQHYTTPPTFGQGTKVEIKGSTSGGGSGGGSGGGGSSEVQLVESGGGLVQPGGSLRLSCAASGFNIKDTYIHWVRQAPGKGLEWVARIYPTNGYTRYADSVKGRFTISADTSKNTAYLQMNSLRAEDTAVYYCSRWGGDGFYAMDYWGQGTLVTVSSESKYGPPCPPCPGGGSSGGGSGGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGKMFWVLVVVGGVLACYSLLVTVAFIIFWVRSKRSRGGHSDYMNMTPRRPGPTRKHYQPYAPPRDFAAYRSGGGRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPRLEGGGEGRGSLLTCGDVEENPGPRMPPPRLLFFLLFLTPMEVRPEEPLVVKVEEGDNAVLQCLKGTSDGPTQQLTWSRESPLKPFLKLSLGLPGLGIHMRPLAIWLFIFNVSQQMGGFYLCQPGPPSEKAWQPGWTVNVEGSGELFRWNVSDLGGLGCGLKNRSSEGPSSPSGKLMSPKLYVWAKDRPEIWEGEPPCVPPRDSLNQSLSQDLTMAPGSTLWLSCGVPPDSVSRGPLSWTHVHPKGPKSLLSLELKDDRPARDMWVMETGLLLPRATAQDAGKYYCHRGNLTMSFHLEITARPVLWHWLLRTGGWKVSAVTLAYLIFCLCSLVGILHLQRALVLRRKR

SEQ ID NO: 32 - protein, 808 aa, synthetic construct
MLLLVTSLLLCELPHPAFLLIPDIQMTQSPSSLSASVGDRVTITCRASQDVNTAVAWYQQKPGKAPKLLIYSASFLYSGVPSRFSGSRSGTDFTLTISSLQPEDFATYYCQQHYTTPPTFGQGTKVEIKGSTSGGGSGGGSGGGGSSEVQLVESGGGLVQPGGSLRLSCAASGFNIKDTYIHWVRQAPGKGLEWVARIYPTNGYTRYADSVKGRFTISADTSKNTAYLQMNSLRAEDTAVYYCSRWGGDGFYAMDYWGQGTLVTVSSGGGSSGGGSGMFWVLVVVGGVLACYSLLVTVAFIIFWVRSKRSRGGHSDYMNMTPRRPGPTRKHYQPYAPPRDFAAYRSGGGRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPRLEGGGEGRGSLLTCGDVEENPGPRMPPPRLLFFLLFLTPMEVRPEEPLVVKVEEGDNAVLQCLKGTSDGPTQQLTWSRESPLKPFLKLSLGLPGLGIHMRPLAIWLFIFNVSQQMGGFYLCQPGPPSEKAWQPGWTVNVEGSGELFRWNVSDLGGLGCGLKNRSSEGPSSPSGKLMSPKLYVWAKDRPEIWEGEPPCVPPRDSLNQSLSQDLTMAPGSTLWLSCGVPPDSVSRGPLSWTHVHPKGPKSLLSLELKDDRPARDMWVMETGLLLPRATAQDAGKYYCHRGNLTMSFHLEITARPVLWHWLLRTGGWKVSAVTLAYLIFCLCSLVGILHLQRALVLRRKR

SEQ ID NO: 33 - protein, 802 aa, synthetic construct
MLLLVTSLLLCELPHPAFLLIPDIQMTQSPSSLSASVGDRVTITCRASQDVNTAVAWYQQKPGKAPKLLIYSASFLYSGVPSRFSGSRSGTDFTLTISSLQPEDFATYYCQQHYTTPPTFGQGTKVEIKGSTSGGGSGGGSGGGGSSEVQLVESGGGLVQPGGSLRLSCAASGFNIKDTYIHWVRQAPGKGLEWVARIYPTNGYTRYADSVKGRFTISADTSKNTAYLQMNSLRAEDTAVYYCSRWGGDGFYAMDYWGQGTLVTVSSGGGSSGGGSGIYIWAPLAGTCGVLLLSLVITKRGRKKLLYIFKQPFMRPVQTTQEEDGCSCRFPEEEEGGCELGGGRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPRLEGGGEGRGSLLTCGDVEENPGPRMPPPRLLFFLLFLTPMEVRPEEPLVVKVEEGDNAVLQCLKGTSDGPTQQLTWSRESPLKPFLKLSLGLPGLGIHMRPLAIWLFIFNVSQQMGGFYLCQPGPPSEKAWQPGWTVNVEGSGELFRWNVSDLGGLGCGLKNRSSEGPSSPSGKLMSPKLYVWAKDRPEIWEGEPPCVPPRDSLNQSLSQDLTMAPGSTLWLSCGVPPDSVSRGPLSWTHVHPKGPKSLLSLELKDDRPARDMWVMETGLLLPRATAQDAGKYYCHRGNLTMSFHLEITARPVLWHWLLRTGGWKVSAVTLAYLIFCLCSLVGILHLQRALVLRRKR

SEQ ID NO: 34 - protein, 658 aa, synthetic construct
DIQMTQSPSSLSASVGDRVTITCRASQDVNTAVAWYQQKPGKAPKLLIYSASFLYSGVPSRFSGSRSGTDFTLTISSLQPEDFATYYCQQHYTTPPTFGQGTKVEIKGSTSGGGSGGGSGGGGSSEVQLVESGGGLVQPGGSLRLSCAASGFNIKDTYIHWVRQAPGKGLEWVARIYPTNGYTRYADSVKGRFTISADTSKNTAYLQMNSLRAEDTAVYYCSRWGGDGFYAMDYWGQGTLVTVSSESKYGPPCPPCPAPEFEGGPSVFLFPPKPKDTLMISRTPEVTCVVVDVSQEDPEVQFNWYVDGVEVHNAKTKPREEQFQSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGKMFWVLVVVGGVLACYSLLVTVAFIIFWVRSKRSRGGHSDYMNMTPRRPGPTRKHYQPYAPPRDFAAYRSGGGRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 35 - protein, 652 aa, synthetic construct
DIQMTQSPSSLSASVGDRVTITCRASQDVNTAVAWYQQKPGKAPKLLIYSASFLYSGVPSRFSGSRSGTDFTLTISSLQPEDFATYYCQQHYTTPPTFGQGTKVEIKGSTSGGGSGGGSGGGGSSEVQLVESGGGLVQPGGSLRLSCAASGFNIKDTYIHWVRQAPGKGLEWVARIYPTNGYTRYADSVKGRFTISADTSKNTAYLQMNSLRAEDTAVYYCSRWGGDGFYAMDYWGQGTLVTVSSESKYGPPCPPCPAPEFEGGPSVFLFPPKPKDTLMISRTPEVTCVVVDVSQEDPEVQFNWYVDGVEVHNAKTKPREEQFQSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGKIYIWAPLAGTCGVLLLSLVITKRGRKKLLYIFKQPFMRPVQTTQEEDGCSCRFPEEEEGGCELGGGRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 36 - protein, 474 aa, synthetic construct
DIQMTQSPSSLSASVGDRVTITCRASQDVNTAVAWYQQKPGKAPKLLIYSASFLYSGVPSRFSGSRSGTDFTLTISSLQPEDFATYYCQQHYTTPPTFGQGTKVEIKGSTSGGGSGGGSGGGGSSEVQLVESGGGLVQPGGSLRLSCAASGFNIKDTYIHWVRQAPGKGLEWVARIYPTNGYTRYADSVKGRFTISADTSKNTAYLQMNSLRAEDTAVYYCSRWGGDGFYAMDYWGQGTLVTVSSAKPTTTPAPRPPTPAPTIASQPLSLRPEACRPAAGGAVHTRGLDFACDIYIWAPLAGTCGVLLLSLVITGGGKRGRKKLLYIFKQPFMRPVQTTQEEDGCSCRFPEEEEGGCELGGGRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 37 - protein, 445 aa, synthetic construct
DIQMTQSPSSLSASVGDRVTITCRASQDVNTAVAWYQQKPGKAPKLLIYSASFLYSGVPSRFSGSRSGTDFTLTISSLQPEDFATYYCQQHYTTPPTFGQGTKVEIKGSTSGGGSGGGSGGGGSSEVQLVESGGGLVQPGGSLRLSCAASGFNIKDTYIHWVRQAPGKGLEWVARIYPTNGYTRYADSVKGRFTISADTSKNTAYLQMNSLRAEDTAVYYCSRWGGDGFYAMDYWGQGTLVTVSSESKYGPPCPPCPGGGSSGGGSGIYIWAPLAGTCGVLLLSLVITKRGRKKLLYIFKQPFMRPVQTTQEEDGCSCRFPEEEEGGCELGGGRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 38 - protein, 552 aa, synthetic construct
DIQMTQSPSSLSASVGDRVTITCRASQDVNTAVAWYQQKPGKAPKLLIYSASFLYSGVPSRFSGSRSGTDFTLTISSLQPEDFATYYCQQHYTTPPTFGQGTKVEIKGSTSGGGSGGGSGGGGSSEVQLVESGGGLVQPGGSLRLSCAASGFNIKDTYIHWVRQAPGKGLEWVARIYPTNGYTRYADSVKGRFTISADTSKNTAYLQMNSLRAEDTAVYYCSRWGGDGFYAMDYWGQGTLVTVSSESKYGPPCPPCPGGGSSGGGSGGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGKIYIWAPLAGTCGVLLLSLVITKRGRKKLLYIFKQPFMRPVQTTQEEDGCSCRFPEEEEGGCELGGGRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 39 - protein, 558 aa, synthetic construct
DIQMTQSPSSLSASVGDRVTITCRASQDVNTAVAWYQQKPGKAPKLLIYSASFLYSGVPSRFSGSRSGTDFTLTISSLQPEDFATYYCQQHYTTPPTFGQGTKVEIKGSTSGGGSGGGSGGGGSSEVQLVESGGGLVQPGGSLRLSCAASGFNIKDTYIHWVRQAPGKGLEWVARIYPTNGYTRYADSVKGRFTISADTSKNTAYLQMNSLRAEDTAVYYCSRWGGDGFYAMDYWGQGTLVTVSSESKYGPPCPPCPGGGSSGGGSGGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGKMFWVLVVVGGVLACYSLLVTVAFIIFWVRSKRSRGGHSDYMNMTPRRPGPTRKHYQPYAPPRDFAAYRSGGGRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 40 - protein, 439 aa, synthetic construct
DIQMTQSPSSLSASVGDRVTITCRASQDVNTAVAWYQQKPGKAPKLLIYSASFLYSGVPSRFSGSRSGTDFTLTISSLQPEDFATYYCQQHYTTPPTFGQGTKVEIKGSTSGGGSGGGSGGGGSSEVQLVESGGGLVQPGGSLRLSCAASGFNIKDTYIHWVRQAPGKGLEWVARIYPTNGYTRYADSVKGRFTISADTSKNTAYLQMNSLRAEDTAVYYCSRWGGDGFYAMDYWGQGTLVTVSSGGGSSGGGSGMFWVLVVVGGVLACYSLLVTVAFIIFWVRSKRSRGGHSDYMNMTPRRPGPTRKHYQPYAPPRDFAAYRSGGGRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 41 - protein, 433 aa, synthetic construct
DIQMTQSPSSLSASVGDRVTITCRASQDVNTAVAWYQQKPGKAPKLLIYSASFLYSGVPSRFSGSRSGTDFTLTISSLQPEDFATYYCQQHYTTPPTFGQGTKVEIKGSTSGGGSGGGSGGGGSSEVQLVESGGGLVQPGGSLRLSCAASGFNIKDTYIHWVRQAPGKGLEWVARIYPTNGYTRYADSVKGRFTISADTSKNTAYLQMNSLRAEDTAVYYCSRWGGDGFYAMDYWGQGTLVTVSSGGGSSGGGSGIYIWAPLAGTCGVLLLSLVITKRGRKKLLYIFKQPFMRPVQTTQEEDGCSCRFPEEEEGGCELGGGRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 42 - protein, 22 aa, synthetic construct
ESKYGPPCPPCPGGGSSGGGSG